SEQUENCE LISTING (6 sequences)

SEQ ID NO: 1 - DNA, 2187 bp, Homo sapiens
  hepatocyte growth factor (HGF)
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 2 - protein, 728 aa, Homo sapiens
  hepatocyte growth factor (HGF)
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 3 - DNA, 4173 bp, Homo sapiens
  hepatocyte growth factor (HGF) receptor
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatag

SEQ ID NO: 4 - protein, 1390 aa, Homo sapiens
  PEPTIDE (0)...(0): hepatocyte growth factor (HGF) receptor
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 5 - DNA, 5181 bp, Artificial Sequence
  mammalian expression plasmid vector pVAX1HGF/MGB1 including human HGF cDNA packaged in HVJ-E vector
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgttaacccgggtcgaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 6 - DNA, 7681 bp, Artificial Sequence
  mammalian expression plasmid vector pcDNA3.1(-)HGF
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgctctagaactagctggatcctgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgttttccagctggtatatggccctgaaggatcagatctggttttaatgaagcttgccaggcctgctgtcdtggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgttaacccgggtcgaagcggccgccactgtgctggatatctgcagaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2016/0271219 A1
(43) Pub. Date: Sep. 22, 2016

(54) LYMPHANGIOGENESIS-PROMOTING AGENTS

(71) Applicant: AnGes MG, Inc., Ibaraki-shi (JP)

(72) Inventors: Ryuichi Morishita, Suita-shi (JP); Yasufumi Kaneda, Minoo-shi (JP); Hironori Nakagami, Ibaraki-shi (JP); Yukihiro Saito, Asahikawa-shi (JP)

(21) Appl. No.: 15/088,443

(22) Filed: Apr. 1, 2016

Related U.S. Application Data
(63) Continuation of application No. 11/996,898, filed on Feb. 3, 2009, now abandoned.

(30) Foreign Application Priority Data
Jul. 28, 2005 (JP) 2005-219410
May 29, 2006 (JP) 2006-148970

(51) Int. Cl. A61K 38/18; G01N 33/68; C12Q 1/02; A61K 48/00

(52) U.S. Cl. CPC A61K 38/1833; A61K 48/0016; A61K 48/0075; G01N 33/6872; C12Q 1/025; G01N 2500/04

(57) ABSTRACT

Described herein is the discovery that HGFs activate the growth and migration of lymphatic endothelial cells and thereby promote lymphangiogenesis. The present invention is based on this finding, and provides lymphangiogenesis-promoting agents comprising as active ingredients HGFs, or proteins or compounds functionally equivalent thereto. Based on the finding described above, the present invention also provides methods for promoting lymphangiogenesis which comprise the step of locally administering HGFs or proteins functionally equivalent thereto to affected areas in patients with lymphedema.

### CROSS-REFERENCES TO RELATED APPLICATIONS

This application is a Continuation of U.S. patent application Ser. No. 11/996,898, filed Feb. 3, 2009, which is a U.S. National Phase of PCT/JP2006/315010, filed Jul. 28, 2006, which claims priority to Japanese Patent Application Nos. 2005-219410, filed Jul. 28, 2005, and 2006-148970, filed May 29, 2006. The contents of all of the aforementioned applications are herein incorporated by reference in their entirety.

### TECHNICAL FIELD

The present invention relates to novel lymphangiogenesis-promoting agents comprising, as active ingredients, HGFs or nucleic acids encoding HGFs, and/or methods for promoting lymphangiogenesis. The present invention also relates to agents or methods for preventing or treating lymphedema. The present invention further relates to methods of screening for compounds having lymphangiogenesis-promoting activity or compounds having preventive or therapeutic effects on lymphedema.

### BACKGROUND ART

Lymphedema refers to a condition characterized by the occlusion of lymphatic vessels which, in turn, causes abnormal congestion of tissue fluid which results in swelling, chronic inflammation, and/or fibrosis. There are primary and secondary lymphedemas. Known instances of primary lymphedema include Milroy's disease, Meige's disease, distal hypoplasia, proximal obstructive lymphadenopathy, and lymphangiectasia. Secondary lymphedema arises from another disease; for example, it often appears as an aftereffect of surgical treatment of cancers. In particular, secondary lymphedema often occurs after surgery or radiation therapy for breast cancer, uterine cancer, prostatic cancer, Kaposi's sarcoma and such. Particularly, after breast cancer surgery, lymphedema often appears in the upper limbs. 80% or more cases of lymphedema of the upper limbs arise after breast cancer surgery. Furthermore, lymphedema of the upper limbs is presumed to develop at a frequency of several percent or more after breast cancer surgery. In contrast, lymphedema of the lower limbs is often observed subsequent to uterine cancer surgery. In addition to the above, infections, injuries such as burn injury, inflammations and the like can also result in secondary lymphedema.

Lymphedema significantly impairs motor functions and increases the risk of infection in the affected areas, both of which result in reduction of patients' QOL. However, common therapies for lymphedema, such as massage, exercise therapy, and wearing a supporter, merely treat symptoms; neither radical treatment methods nor therapeutic agents are available at present and almost no therapeutic agent is known. While a compound called “guaifenesin” has been reported to be effective in treating lymphedema, its therapeutic effect still remains unclear (Patent Document 1).

VEGF-C, a member of the VEGF family, has been reported to be a peptidic factor that enhances lymphangiogenesis (Patent Documents 2 and 3; Non-Patent Documents 1 and 2). However, the other VEGF family members have no lymphangiogenesis-promoting activity. VEGF-C is the only lymphangiogenesis-promoting factor that is currently known.

Meanwhile, VEGF₁₆₅ (also referred to as “VEGF-A” or more simply “VEGF”; hereinafter also referred to as “VEGF”), is a member of the VEGF family that, like VEGF-C, is also well known as an angiogenic factor; the factor has been also known as a factor that enhances vascular permeability (for example, see Senger, D. et al., Science 219, 983-6 (1983)). In fact, VEGF has been reported to induce edema by its vascular permeability-enhancing activity when overexpressed in tissues (for example, see Isner, J M., et al., Circ. Res. 89: 389-400 (2001)). In fact, edema has been reported to be observed at a frequency of 30% or more in human patients with ischemic diseases of lower limbs when the VEGF gene is intraarterially or intramuscularly introduced into the ischemic areas in lower limbs for the therapeutic purpose (Baumgartner, I. et al., Ann. Intern. Med. 132: 880-884 (2000)). In addition, there are many reports of the increased risk of edema accompanying VEGF gene therapy, including a report showing that edema of the lower limbs was observed in six of nine patients in which a plasmid encoding naked VEGF was administered to ischemic areas of the lower limbs by intramuscular injection (Baumgartner, I. et al., Circulation 97: 1114-1123 (1998)) and another report showing that edema appeared in three limbs when a plasmid encoding naked VEGF was intramuscularly administered to ischemic areas in seven limbs of six patients with lower limb ischemia due to thromboangiitis obliterans (TAO) (Isner, J M., et al., J. Vasc. Surg. 28: 964-975 (1998)).

In addition, VEGF-C is a ligand for VEGF receptor 3 (VEGFR-3), and the signal from VEGFR-3 alone is known to be sufficient for lymphangiogenesis. VEGF-C also binds to VEGFR-2. Since VEGFR-2-deficient mice die earlier than VEGF-A-deficient mice, VEGF-C and other VEGF members are presumed to be able to complement the VEGF activity (Scavelli, C. et al., J. Anat. 433-449 (2004)). VEGF exerts its function through binding to VEGFR-1 and VEGFR-2. Thus, the activation of VEGFR-2 is thought to induce the enhancement of vascular permeability (for example, see Issbrucker, K. et al., FASEB J. express article 10.1096/fj.02-0329fje. Published online Dec. 18, 2002).

HGF is hepatocyte growth factor, and has not only the activity of promoting the growth of hepatocytes but also other various physiological activities, including angiogenic activity (for example, see Non-Patent Document 3). HGFs are presently applied to the treatment of ischemic disease based on their angiogenic activity (Patent Documents 4 and 5; Non-Patent Document 4).

[Patent Document 1] WO03/000242

[Patent Document 2] U.S. Pat. No. 6,818,220

[Patent Document 3] U.S. Pat. No. 6,689,352

[Patent Document 4] WO97/07824

[Patent Document 5] WO01/32220

[Non-patent Document 1] Szuba, A. et al., FASEB J. express article 10.1096/fj.02-0401fje. Published online Oct. 18, 2002

[Non-patent Document 2] Young-sup, Yoo et al., J. Clin. Invest. 111:717-725 (2003)

[Non-patent Document 3] Nakamura Y. et al., J Hypertens. Sep. 14(9):1067-72 (1996)

[Non-patent Document 4] Taniyama Y., et al., Gene Therapy 8, 181-189 (2001)

### DISCLOSURE OF THE INVENTION

### Problems to be Solved by the Invention

The present invention was achieved in view of the circumstances described above. An objective of the present invention is to provide novel uses of HGFs and genes encoding the same. More specifically, an objective of the present invention is to provide novel therapeutic agents and treatment methods for lymphedema, a condition for which no effective therapeutic agent or treatment method is presently available.

### Means for Solving the Problems

The present inventors conducted dedicated studies to achieve the objectives described above. The present inventors searched for factors that could reduce or eliminate lymphedema, and found that HGFs significantly reduced lymphedema. Then, the present inventors discovered that HGFs had lymphangiogenic activity to induce the growth of lymphatic endothelial cells.

As described above, there are many reports associating VEGF gene therapy with an increased risk of inducing edema. Thus, the present inventors considered that VEGF to be unsuitable for lymphedema treatment. Furthermore, since it is suggested that VEGF-C may enhance cell permeability by binding to VEGF receptors other than VEGFR-3, the present inventors considered that the application of VEGF-C to lymphedema treatment also constituted a high risk.

HGF has been known as an angiogenic factor. VEGF is also known as an angiogenic factor, but is presumed to have neither lymphangiogenic activity (see, for example, Dev. Biol. 1997, 188: 96-109) nor the effect of reducing lymphedema, as described above. In view of the above, the novel findings by the present inventors that HGFs have lymphangiogenic activity and the effect of reducing lymphedema is a remarkable discovery.

To confirm the lymphangiogenic activity of HGFs based on its action mechanism, the present inventors demonstrated that the HGF receptor c-met was expressed in lymphatic endothelial cells and that phosphorylation of MAPK and Akt, which are intracellular signaling proteins whose phosphorylation is known to be induced by HGFs, was also induced in lymphatic endothelial cells.

These findings demonstrate that HGFs and their genes are effective as therapeutic or preventive agents for lymphedema, and as lymphangiogenesis-promoting agents. Specifically, the present invention provides the following [1] to [44]:

[1] a lymphangiogenesis-promoting agent comprising as an active ingredient an HGF or a protein or compound functionally equivalent to an HGF;

[2] the agent of [1], wherein the HGF or protein functionally equivalent to an HGF is any one of the following (a) to (d):

(a) a protein comprising the amino acid sequence of SEQ ID NO: 2,

(b) a protein encoded by a nucleic acid comprising the coding sequence in the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[3] a lymphangiogenesis-promoting agent, which comprises as an active ingredient a nucleic acid encoding an HGF or a protein functionally equivalent to an HGF;

[4] the agent of [3], wherein the nucleic acid encoding an HGF or a protein functionally equivalent to an HGF is a nucleic acid encoding any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[5] the agent of [3] or [4], wherein the nucleic acid has been inserted into a mammalian expression vector;

[6] the agent of any one of [3] to [5], wherein the nucleic acid is a naked nucleic acid;

[7] the agent of any one of [1] to [6], which is used as a pharmaceutical agent for preventing or treating lymphedema;

[8] a method for promoting lymphangiogenesis, which comprises the step of administering to a subject an HGF or a protein or compound functionally equivalent to an HGF;

[9] the method of [8], wherein the HGF or protein functionally equivalent to an HGF is any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[10] a method for promoting lymphangiogenesis, which comprises the step of administering to a subject a nucleic acid encoding an HGF or a protein functionally equivalent to an HGF;

[11] the method of [10], wherein the nucleic acid encoding an HGF or a protein functionally equivalent to an HGF is a nucleic acid encoding any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[12] the method of [10] or [11], wherein the nucleic acid has been inserted into a mammalian expression vector;

[13] the method of any one of [10] to [12], wherein the nucleic acid is a naked nucleic acid;

[14] a method for inducing activation of an HGF receptor and promoting lymphangiogenesis through the activation;

[15] a method for preventing or treating lymphedema, which comprises the step of administering to a subject an HGF or a protein or compound functionally equivalent to an HGF;

[16] the method of [15], wherein the HGF or protein functionally equivalent to an HGF is any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[17] a method for preventing or treating lymphedema, which comprises the step of administering to a subject a nucleic acid encoding an HGF or a protein functionally equivalent to an HGF;

[18] the method of [17], wherein the nucleic acid encoding an HGF or a protein functionally equivalent to an HGF is a nucleic acid encoding any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[19] the method of [17] or [18], wherein the nucleic acid has been inserted into a mammalian expression vector;

[20] the method of any one of [17] to [19], wherein the nucleic acid is a naked nucleic acid;

[21] use of an HGF or a protein or compound functionally equivalent to an HGF for producing a lymphangiogenesis-promoting agent or a pharmaceutical agent to be used to prevent or treat lymphedema;

[22] the use of [21], wherein the HGF or protein functionally equivalent to an HGF is any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[23] use of a nucleic acid encoding an HGF or a protein functionally equivalent to an HGF for producing a lymphangiogenesis-promoting agent or a pharmaceutical agent to be used to prevent or treat lymphedema;

[24] the use of [23], wherein the nucleic acid encoding an HGF or a protein functionally equivalent to an HGF is a nucleic acid encoding any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[25] the use of [23] or [24], wherein the nucleic acid has been inserted into a mammalian expression vector;

[26] the use of any one of [23] to [25], wherein the nucleic acid is a naked nucleic acid;

[27] a method of screening for a compound having lymphangiogenesis-promoting activity or a compound having an effect of preventing or treating lymphedema, wherein the method comprises the following steps:

(a) contacting a test compound with an HGF receptor or a protein functionally equivalent to an HGF receptor,

(b) detecting the binding between the protein and test compound, and

(c) selecting a test compound that binds to the protein,

[28] the method of [27], wherein the HGF receptor or protein functionally equivalent to an HGF receptor is selected from the following (i) to (iv):

(i) a protein comprising the amino acid sequence of SEQ ID NO: 4,

(ii) a protein encoded by a nucleic acid comprising the coding region of the nucleotide sequence of SEQ ID NO: 3,

(iii) a protein comprising an amino acid sequence with a substitution, deletion, insertion, and/or addition of one or more amino acids in the amino acid sequence of SEQ ID NO: 4, which is functionally equivalent to a protein comprising the amino acid sequence of SEQ ID NO: 4, and

(iv) a protein encoded by a nucleic acid that hybridizes under stringent conditions to a nucleic acid comprising the nucleotide sequence of SEQ ID NO: 3, which is functionally equivalent to a protein comprising the amino acid sequence of SEQ ID NO: 4;

[29] a method of screening for a compound having lymphangiogenesis-promoting activity or a compound having an effect of preventing or treating lymphedema, wherein the method comprises the following steps:

(a) contacting a test compound with a cell expressing an HGF receptor or a protein functionally equivalent to an HGF receptor,

(b) measuring the growth capacity or migratory activity of the cell, or phosphorylation of a signaling molecule, and

(c) selecting a test compound that increases the growth capacity or migratory activity of the cell, or causes phosphorylation of the signaling molecule, as compared to when the test compound is not contacted;

[30] the method of [29], wherein the HGF receptor or protein functionally equivalent to an HGF receptor is selected from the following (i) to (iv):

(i) a protein comprising the amino acid sequence of SEQ ID NO: 4,

(ii) a protein encoded by a nucleic acid comprising the coding region of the nucleotide of SEQ ID NO: 3,

(iii) a protein comprising an amino acid sequence with a substitution, deletion, insertion, and/or addition of one or more amino acids in the amino acid sequence of SEQ ID NO: 4, which is functionally equivalent to a protein comprising the amino acid sequence of SEQ ID NO: 4, and

(iv) a protein encoded by a nucleic acid that hybridizes under stringent conditions to a nucleic acid comprising the nucleotide sequence of SEQ ID NO: 3, which is functionally equivalent to a protein comprising the amino acid sequence of SEQ ID NO: 4;

[31] a vector for preventing or treating lymphedema, which is a mammalian expression vector into which a nucleic acid encoding an HGF or a protein functionally equivalent to an HGF has been inserted;

[32] the vector of [31], wherein the nucleic acid encoding an HGF or the protein functionally equivalent to an HGF is a nucleic acid encoding any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[33] the vector of [31] or [32], which comprises the nucleotide sequence of SEQ ID NO: 5 or 6;

[34] the vector of any one of [31] to [33], which is administered in a naked state;

[35] the vector of any one of [31] to [34], which is administered by intramuscular injection to or around an affected area in a subject;

[36] a pharmaceutical agent for preventing or treating lymphedema, which comprises as an active ingredient a mammalian expression vector into which a nucleic acid encoding an HGF or a protein functionally equivalent to an HGF has been inserted, wherein the vector is administered in a naked state by intramuscular injection to or around an affected area in a subject;

[37] the pharmaceutical agent of [36], wherein the nucleic acid encoding an HGF or a protein functionally equivalent to an HGF is a nucleic acid encoding any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[38] the pharmaceutical agent of [36] or [37], wherein the vector comprises the nucleotide sequence of SEQ ID NO: 5 or 6;

[39] a method for preventing or treating lymphedema, which comprises the step of administering, in a naked state, a mammalian expression vector into which a nucleic acid encoding an HGF or a protein functionally equivalent to an HGF has been inserted, to or around an affected area in a subject by intramuscular injection;

[40] the method of [39], wherein the nucleic acid encoding an HGF or a protein functionally equivalent to an HGF is a nucleic acid encoding any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2;

[41] the method of [40], wherein the vector comprises the nucleotide sequence of SEQ ID NO: 5 or 6;

[42] use of a mammalian expression vector into which a nucleic acid encoding an HGF or a protein functionally equivalent to an HGF has been inserted, for producing a pharmaceutical agent for preventing or treating lymphedema, wherein the vector is administered in a naked state to or around an affected area in a subject by intramuscular injection;

[43] the use of [42], wherein the nucleic acid encoding an HGF or a protein functionally equivalent to an HGF is a nucleic acid encoding any one of the following (a) to (d):

(a) a protein comprising the amino acid sequence of SEQ ID NO: 2,

(b) a protein encoded by a nucleic acid comprising the coding region of the nucleotide sequence of SEQ ID NO: 1,

(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2, and

(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2; and

[44] the use of [42] or [43], wherein the vector comprises the nucleotide sequence of SEQ ID NO: 5 or 6.

### BRIEF DESCRIPTION OF THE DRAWINGS

FIG. 1 depicts a series of photographs showing the results of immunofluorescence staining of lymphatic endothelial cells. The top panels correspond to staining with anti-c-met antibody; the middle panels correspond to nuclear staining with DAPI; and the bottom panels constitute merged images of the two. The results confirm that all cells expressed c-met.

FIG. 2 is a graph depicting the results on an MTS assay examining the effect of a recombinant human HGF on growth capacity. The vertical axis indicates the measured values. The results demonstrate that the HGF promotes the growth of lymphatic endothelial cells in a concentration-dependent manner.

FIG. 3 is a graph depicting the results of a migration assay examining the effect of a recombinant human HGF on migratory capacity. The vertical axis indicates the number of cells. The results demonstrate that the HGF promotes the migration of lymphatic endothelial cells.

FIG. 4 is a graph depicting the results of an MTS assay examining the effect of introducing a naked cDNA plasmid on growth capacity. The vertical axis indicates the measured values. The results demonstrate that the introduction of the HGF gene is effective to promote the growth of lymphatic endothelial cells.

FIG. 5 is a graph depicting the results of a c-fos promoter assay examining the effect of introducing a naked cDNA plasmid on growth capacity. The vertical axis indicates the measured values for luciferase activity. The results demonstrate that the introduction of the HGF gene is effective to promote the growth of lymphatic endothelial cells.

FIG. 6 is a graph depicting the results of a c-fos promoter assay examining the effect of introducing a naked cDNA plasmid on growth capacity. The vertical axis indicates the measured values for luciferase activity. These results demonstrate that the expression vector for HGF cDNA significantly enhances the c-fos promoter activity while the expression vector for VEGF cDNA does not enhance the c-fos promoter activity.

FIGS. 7A and 7B show a series of graphs demonstrating the effects of HGF on the growth and migratory activity of human lymphatic endothelial cells. FIG. 7A depicts the results of an MTS assay examining cell growth; and FIG. 7B depicts the results of a migration assay examining migratory activity. Each assay was carried out after the recombinant human HGF was added at a concentration of 0, 2, 10, or 50 ng/ml to the culture media of human lymphatic endothelial cells. In FIG. 7A, the vertical axis indicates the absorbance at 490 nm; and in FIG. 7B the vertical axis indicates the number of cells. These results demonstrate that the addition of HGF promotes the growth and migratory activity. *p<0.001 (relative to 0 ng/ml of the recombinant human HGF); †p<0.001 (relative to 2 ng/ml of the recombinant human HGF).

FIG. 8 is a graph depicting the lymphedema-improving effect of the expression plasmid for HGF cDNA in rat models of lymphedema which have lymphedema at the base of their tail. The horizontal axis indicates time elapsed after surgery (days), and the vertical axis indicates the thickness of the base of tail (mm). The Venus group as a control, HGF group, and VEGF group were injected with 0.1 ml of 200 μg expression plasmids for GFP, HGF, and VEGF, respectively. The physiological saline group was injected with 0.1 ml of physiological saline. The results demonstrate that the reduction of tail thickness is promoted by introducing the naked HGF cDNA and thus lymphedema is improved.

FIG. 9 is a graph obtained by calculating the area under the curve of the graph shown in FIG. 8. The results demonstrate that the tail thickness was significantly reduced only in the group of rats into which the HGF gene was introduced. The vertical axis indicates the area ratio (%) relative to the control. ‡p<0.0001 (relative to each of the group introduced with the VEGF gene, the Venus group, the physiological saline group, and the group treated with surgery alone).

FIGS. 10A and 10B show a series of photographs and graphs depicting the results of examination on the lymphangiogenesis at the surgical sites after the introduction of the HGF or VEGF gene in rat models of lymphedema. FIG. 10A is composed of micrographs of immunostained sections of the tissue near the gene injection site in each rat. Immunostaining was carried out using antibodies against PECAM-1, LYVE-1, Prox1, and c-Met. The top panels are from rats into which the HGF gene was introduced; the middle panels are from rats into which the VEGF gene was introduced; and the bottom panels are from control rats injected with physiological saline. FIG. 10B is composed of graphs depicting the number of vessels that were positive for immunostaining with each antibody. HGF: rats into which the HGF gene was introduced; VEGF: rats into which the VEGF gene was introduced; control: rats injected with physiological saline. The vertical axis indicates the number of positive vessels. These results demonstrate that the number of vessels positive for the lymphatic endothelial cell markers (LYVE-1 and Prox1) is significantly increased only in the rats introduced with the HGF gene; whereas the number of vessels positive for the endothelial cell marker PECAM-1 is increased in both the rats introduced with the HGF gene and the rats introduced with the VEGF gene as compared to the control, but there is no significant difference. Furthermore, it is shown that the number of vessels positive for c-Met in the rats introduced with the HGF gene is significantly increased as compared with that in the rats introduced with the VEGF gene and tends to be increased as compared to the control.

FIGS. 11A and 11B show a series of photographs depicting phosphorylation of MAPK and Akt after HGF stimulation of canine thoracic duct-derived lymphatic endothelial cells. FIGS. 11A and 11B depict results of Western blotting using antibodies specific to phosphorylated MAPK and phosphorylated Akt, respectively (each upper panel). The lower panels show results of Western blotting using antibodies specific to MAPK and Akt regardless of their phosphorylation state. These antibodies were used as internal controls to demonstrate that samples contain almost equal amounts of MAPK or Akt.

### BEST MODE FOR CARRYING OUT THE INVENTION

The present invention relates to lymphangiogenesis-promoting agents comprising, as active ingredients HGFs, or proteins or compounds (hereinafter sometimes referred to as “HGF”) functionally equivalent thereto. The present invention is based on the above-mentioned finding of the present inventors that HGFs activate the growth and migration of lymphatic endothelial cells and thereby promote lymphangiogenesis.

The present invention is further based on the finding that HGFs activate the growth and migration of lymphatic endothelial cells isolated from neither fetal nor neonatal but adult animals and thereby promote lymphangiogenesis. Since lymphatic endothelial cells are differentiated from fetal veins at an early stage of embryogenesis, lymphatic endothelial cell marker proteins are also expressed in venous endothelial cells at the early fetal stage. Lymphatic endothelial cells are also known to further differentiate into venous endothelial cells (Wigle J T and Oliver G. Cell, 98: 769-778 (1999)). Therefore, fetal or neonatal cells are highly likely to be at a stage before terminal differentiation even when expressing lymphatic endothelial cell markers. Thus, fetal or neonatal lymphatic endothelial cells are unlikely to accurately reflect lymphatic endothelial cells of adult animals. The present invention demonstrated the effect of HGFs on lymphatic endothelial cells of adult animals. Thus, HGFs and their genes were found to be effective as therapeutic agents for patients (more particularly adults) suffering from lymphedema after the surgical removal of cancer tissues and/or lymph nodes for cancer treatment and for other lymphedema patients.

In one embodiment of the present invention, HGFs include proteins comprising the amino acid sequence of SEQ ID NO: 2. In another embodiment of the present invention, HGFs include proteins encoded by the “HGF gene”. Such a gene includes, for example, nucleic acids comprising the coding region of the nucleotide sequence of SEQ ID NO: 1.

The term “protein functionally equivalent to an HGF” in the context of the present invention encompasses proteins isolated from humans or nonhuman animals which have a biological activity equivalent to that of a protein comprising the amino acid sequence of SEQ ID NO: 2. For example, in a more specific embodiment, the proteins functionally equivalent to HGFs in the present invention include proteins comprising an amino acid sequence with a substitution, deletion, insertion, and/or addition of one or more amino acids in the amino acid sequence of SEQ ID NO: 2; and proteins encoded by nucleic acids that hybridize under stringent conditions to nucleic acids comprising the nucleotide sequence of SEQ ID NO: 1, which are functionally equivalent to proteins comprising the amino acid sequence of SEQ ID NO: 2.

HGFs in the present invention may be human or nonhuman HGFs; however, human HGFs are most preferred. Those skilled in the art can obtain the amino acid sequences of nonhuman HGFs and the nucleotide sequences encoding them from various databases.

Furthermore, “proteins and compounds functionally equivalent to HGFs” can also include proteins and compounds that induce intracellular signaling by binding to c-met, an

HGF receptor, and then activating intramolecular kinases of c-met. Thus, such proteins and compounds also include HGF agonists, for example, antibodies agonistically acting on c-met, which are disclosed in Prat, M., et al. (J. Cell Sci. 111 237-247 (1998)), and HGF mutants that bind to HGF receptors, which are disclosed in WO 98/51798.

Biological activity equivalent to that of a protein comprising the amino acid sequence of SEQ ID NO: 2 includes phosphorylation of tyrosines (Y¹³⁴⁹ and Y¹³⁵⁶) in the cytoplasmic domain of c-met and/or phosphorylation of signaling molecules downstream thereof (see, for example, Graziani, A. et al., J. Biol. Chem. 266, 22087-22090 (1991); Graziani, A. et al., J. Biol. Chem. 268, 9165-9168 (1993); Nakagami, H. et al., Hypertension, 37 [part2]: 581-586 (2001)), and promotion of growth and migration of vascular endothelial cells and/or lymphatic endothelial cells; but is not limited thereto. The activity includes all biological activities of HGFs on cells.

The “proteins and compounds functionally equivalent to HGFs” in the present invention include all proteins and compounds having such an activity. Whether such compounds have a biological activity equivalent to that of HGFs can be determined by methods known to those skilled in the art. See, for example, Graziani, A. et al., J. Biol. Chem. 266, 22087-22090 (1991); Graziani, A. et al., J. Biol. Chem. 268, 9165-9168 (1993); and Nakagami, H. et al., Hypertension 37[part2]: 581-586 (2001). Such determination as described above can be achieved by methods for assaying the growth capacity, migratory activity, and c-fos promoter activity of lymphatic endothelial cells herein described in the Examples; however, the methods are not limited thereto. Such methods also include methods in which cells expressing c-met are contacted with an HGF and then the phosphorylation of c-met or other downstream molecules in the HGF-c-met signaling pathway is detected in lysates of the cells. These methods are known to those skilled in the art. Furthermore, those skilled in the art can readily make appropriate modifications and improvements to known methods.

For example, HGF production in cells is promoted through the increase in the intracellular cyclic AMP concentration, and as a result the biological activity of HGFs is enhanced (Morishita R., et al., Diabetologia 40 (9):1053-61 (1997)). Accordingly, compounds and proteins such as cilostazol, a type 3 phosphodiesterase inhibitor, that increase the intracellular cyclic AMP concentration are also encompassed in the “proteins and compounds functionally equivalent to HGFs” of the present invention. In addition, antagonists to angiotensin II (Nakano N., et al., Hypertension 32(3): 444-51 (1998)) that suppress HGF production (angiotensin II receptor antagonists) are also included.

Stringent hybridization conditions necessary to isolate the above-described nucleic acids that hybridize under stringent conditions to a nucleic acid comprising the nucleotide sequence of SEQ ID NO: 1 include the conditions of 6 M urea, 0.4% SDS, 0.5x SSC, and 37° C., and hybridization conditions of stringency equivalent thereto. Nucleic acids with higher homology can be expected to be isolated through use of more stringent conditions, for example, the conditions of 6 M urea, 0.4% SDS, 0.1x SSC, and 42° C. The sequences of isolated nucleic acids can be determined by the known methods described below. Homology of isolated nucleic acids over the entirety of their nucleotide sequences is at least 50% or higher, preferably 70% or higher, more preferably 90% or higher (for example, 95%, 96%, 97%, 98%, or 99% or higher) sequence identity.

As an alternative to the above-described methods using hybridization techniques, nucleic acids which hybridize under stringent conditions to nucleic acids comprising the nucleotide sequence of SEQ ID NO: 1, and which encode proteins functionally equivalent to proteins comprising the amino acid sequence of SEQ ID NO: 2, can be isolated by gene amplification method, for example, polymerase chain reaction (PCR) method, using primers synthesized based on the sequence information of a nucleic acid encoding an HGF (SEQ ID NO: 1).

Methods well known to those skilled in the art for preparing proteins functionally equivalent to a certain protein, include methods for introducing mutations into proteins. For example, those skilled in the art can prepare mutants functionally equivalent to HGFs by introducing appropriate mutations into the amino acid sequence of a human HGF using site-directed mutagenesis (Hashimoto-Gotoh, T, Mizuno, T, Ogasahara, Y, and Nakagawa, M. (1995) An oligodeoxyribonucleotide-directed dual amber method for site-directed mutagenesis. Gene 152, 271-275; Zoller, M J, and Smith, M. (1983) Oligonucleotide-directed mutagenesis of DNA fragments cloned into M13 vectors. Methods Enzymol. 100, 468-500; Kramer, W, Drutsa, V, Jansen, HW, Kramer, B, Pflugfelder, M, and Fritz, H J (1984) The gapped duplex DNA approach to oligonucleotide-directed mutation construction. Nucleic Acids Res. 12, 9441-9456; Kramer W, and Fritz H J (1987) Oligonucleotide-directed construction of mutations via gapped duplex DNA Methods. Enzymol. 154, 350-367; Kunkel, T A (1985) Rapid and efficient site-specific mutagenesis without phenotypic selection. Proc Natl Acad Sci USA. 82, 488-492) or the like. Amino acid mutations in proteins may also occur naturally. Thus, the proteins of the present invention also include proteins comprising an amino acid sequence with one or more amino acid mutations in the amino acid sequence of an HGF (SEQ ID NO: 2), which are functionally equivalent to HGFs.

When an amino acid residue is altered, the amino acid is preferably mutated to a different amino acid(s) that conserves the properties of the amino acid side-chain. Examples of amino acid side chain properties are the following: hydrophobic amino acids (A, I, L, M, F, P, W, Y, and V), hydrophilic amino acids (R, D, N, C, E, Q, G, H, K, S, and T), amino acids having aliphatic side chains (G, A, V, L, I, and P), amino acids having hydroxyl group-containing side chains (S, T, and Y), amino acids having sulfur-containing side chains (C and M), amino acids having carboxylic acid- and amide-containing side chains (D, N, E, and Q), amino acids having basic side chains (R, K, and H), and amino acids having aromatic side chains (H, F, Y, and W) (amino acids are represented by one-letter codes in parentheses). Amino acid substitutions within each group are called conservative substitutions. It is already known that a polypeptide comprising a modified amino acid sequence in which one or more amino acid residues in a given amino acid sequence are deleted, added, and/or substituted with other amino acids can retain the original biological activity (Mark, D. F. et al., Proc. Natl. Acad. Sci. USA (1984) 81: 5662-6; Zoller, M. J. and Smith, M., Nucleic Acids Res. (1982) 10: 6487-500; Wang, A. et al., Science (1984) 224: 1431-3; Dalbadie-McFarland, G. et al., Proc. Natl. Acad. Sci. USA (1982) 79: 6409-13). Such mutants have at least 70% amino acid sequence identity to the amino acid sequence of an HGF of the present invention, more preferably at least 75%, even more preferably at least 80%, still more preferably at least 85%, yet more preferably at least 90%, and most preferably at least 95% amino acid sequence identity thereto. Herein, sequence identity is defined as the percentage of residues identical to those in the original amino acid sequence of HGF, determined after the sequences are aligned as needed and gaps are appropriately introduced to maximize the sequence identity. The identity of amino acid sequences can be determined by the method described above.

Nucleotide sequence identity and amino acid sequence identity can be determined using the algorithm BLAST, by Karlin and Altschul (Proc. Natl. Acad. Sci. USA (1993) 90, 5873-7). Programs such as BLASTN and BLASTX were developed based on this algorithm (Altschul et al., J. Mol. Biol. (1990) 215, 403-10). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known (see the website of the National Center for Biotechnology Information (NCBI), Basic Local Alignment Search Tool (BLAST); on the world wide web at ncbi.nlm.nih.gov).

HGFs of the present invention can be isolated from natural sources including various biological samples, for example, cells or tissues expressing HGFs, based on their physical properties and the like. Alternatively, HGFs may be chemically synthesized based on known sequence information. In addition, HGFs can be obtained by using genetic recombination techniques to transform host cells with vectors carrying the genes encoding HGFs, then culturing the resulting transformed cells that produce the recombinant HGFs, and collecting the HGFs from the cells or culture supernatant.

Vectors suitable for producing HGFs using genetic engineering methods include various vectors using viruses, cosmids, plasmids, bacteriophages, and the like (Molecular Cloning 2^{nd} ed., Cold Spring Harbor Press (1989); Current Protocols in Molecular Biology, John Wiley & Sons (1987)). Such vectors include appropriate regulatory sequences, and an HGF-encoding nucleic acid is inserted so as to maintain the correct reading frame relative to the regulatory sequence, so that the HGF is expressed when the vectors are introduced into desired host cells. Any nucleic acids encoding HGF can be used, so long as they can be expressed by the selected vector and host. Such nucleic acids preferably include cDNAs; however, RNAs or the like may be used in some cases. When the host cell is a prokaryotic cell, the “regulatory sequence” includes a promoter, a ribosome-binding site, and a terminator. Alternatively, when the host is a eukaryotic cell, the regulatory sequence includes a promoter and terminator, and as necessary, an enhancer, splicing signal, transcription factor, transactivator, poly A signal and/or polyadenylation signal, and so on. Such expression vectors for HGFs may include selection markers for easily selecting transformed host cells, as necessary. Furthermore, signal peptides may be inserted into vectors to be attached to HGFs so that intracellularly expressed HGFs are translocated into the lumen of the endoplasmic reticulum or translocated extracellularly, or alternatively translocated into the periplasm when the host cells are Gram-negative bacteria. Such signal peptides may be inherent in HGFs or may be derived from different proteins, as long as they are properly recognized in selected host cells. Furthermore, linkers, start codons, stop codons and such may be added, if required.

Genes can be inserted into vectors by ligase reactions using restriction enzyme sites (Molecular Cloning 2^{nd} ed., Cold Spring Harbor Press (1989) Section 5.61-5.63; Current Protocols in Molecular Biology, John Wiley & Sons (1987) 11.4-11.11). Such vectors may be designed by considering codon usage in the host cells to be used, and selecting nucleotide sequences that allow high efficiency expression (Grantham et al., Nucleic Acids Res. (1981) 9, r43-74).

When vectors are introduced into adequate hosts to produce HGFs, the above expression vectors and appropriate hosts can be used in combination. Animal cells, plant cells, and fungal cells may be used as eukaryotic host cells.

Host cells can be transformed using methods suitable for selected hosts and vectors.

For example, when prokaryotic cells are used as the hosts, known methods include calcium treatment and electroporation. Examples also include the Agrobacterium method for plant cells, and the calcium phosphate precipitation method for mammalian cells. The present invention is not particularly limited to these methods. Rather, the present invention can use various known methods, including nuclear microinjection, cell fusion, electroporation, protoplast fusion, lipofectamine methods (GIBCO BRL), DEAF-dextran methods, and methods using FuGENE6 reagent (Boehringer-Mannheim).

Host cells can be cultured by known methods suitable for selected cells. For example, when animal cells are used as the host, the cells may be cultured using a medium such as DMEM, MEM, RPMI-1640, 199, or IMDM, if required, supplemented with fetal calf serum (FCS) and such, at pH of about 6 to 8 at 30° C. to 40° C. for about 15 to 200 hours.

HGFs are preferably used after purification by known methods. HGFs can be purified to homogeneity by conventional protein purification methods. HGFs can be separated and purified, for example, by appropriately selecting and combining chromatographic columns, filters, ultrafiltration, salting out, dialysis, preparative polyacrylamide gel electrophoresis, isoelectric focusing and such (Strategies for Protein Purification and Characterization, A Laboratory Course Manual, Daniel R. Marshak et al., eds., Cold Spring Harbor Laboratory Press (1996)), but the present invention is not limited thereto.

Furthermore, as described above, HGFs of the present invention also include proteins and polypeptides in which amino acid residues are added to HGFs. Examples of proteins and polypeptides in which amino acid residues are added to HGFs include fusion proteins. To prepare a polynucleotide encoding such a fusion protein, for example, a DNA encoding an HGF may be linked in frame with a nucleic acid encoding another protein or polypeptide. The protein or polypeptide to be fused with an HGF is not particularly limited. Any nucleic acid encoding an appropriate protein or polypeptide may be linked depending on the purposes.

The present invention also relates to novel uses of the following proteins, nucleic acids, or compounds:
(1) HGFs, or proteins or compounds that are functionally equivalent thereto;
(2) nucleic acids encoding HGFs or proteins functionally equivalent thereto;
(3) nucleic acids encoding HGFs or proteins functionally equivalent thereto, which are inserted into mammalian expression vectors; and
(4) naked nucleic acids encoding HGFs or proteins functionally equivalent thereto.

Specifically, the present invention relates to the following:

lymphangiogenesis-promoting agents that comprise as active ingredients the proteins, nucleic acids, or compounds listed in (1) to (4) above;

methods for preventing or treating lymphedema, which comprise the step of administering the proteins, nucleic acids, or compounds listed in (1) to (4) above to subjects; and

uses of the proteins, nucleic acids, and compounds listed in (1) to (4) above for producing lymphangiogenesis-promoting agents or agents used to prevent or treat lymphedema.

In this context, HGFs are preferably human HGFs.

The lymphangiogenesis-promoting agents of the present invention comprise as active ingredients the HGFs obtained as described above. The term “comprising an HGF as an ingredient” means comprising an HGF as at least one active ingredient, and content need not be limited. Furthermore, the lymphangiogenesis-promoting agents of the present invention may comprise other active ingredients that promote lymphangiogenesis, in addition to HGFs.

HGFs of the present invention can be formulated according to standard methods (see, for example, Remington's Pharmaceutical Science, latest edition, Mark Publishing Company, Easton, USA), and may comprise pharmaceutically acceptable carriers and/or additives. For example, the following can be comprised: detergents (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the lymphangiogenesis-promoting agents of the present invention are not limited thereto, and may comprise other appropriate conventional carriers. Specific examples of such carriers include light anhydrous silicic acid, lactose, crystalline cellulose, mannitol, starch, carmelose calcium, carmelose sodium, hydroxypropylcellulose, hydroxypropylmethylcellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, and inorganic salt. The agents may also comprise other low-molecular-weight polypeptides; proteins such as serum albumin, gelatin, and immunoglobulin; and amino acids such as glycine, glutamine, asparagine, arginine, and lysine. When the agents are prepared as aqueous solutions for injection, HGFs are dissolved in isotonic solutions containing, for example, physiological saline, dextrose, and other adjuvants including, for example, D-sorbitol, D-mannose, D-mannitol, and sodium chloride. In addition, appropriate solubilizing agents such as alcohols (for example, ethanol), polyalcohols (for example, propylene glycol and PEGs), and non-ionic detergents (polysorbate 80 and HCO-50).

If necessary, HGFs may be encapsulated in microcapsules (e.g., microcapsules made of hydroxymethylcellulose, gelatin, and polymethylmethacrylate), and made into components of colloidal drug delivery systems (e.g. liposomes, albumin microspheres, microemulsions, nanoparticles, and nanocapsules) (for example, see “Remington's Pharmaceutical Science 16th edition”, Oslo Ed. (1980)). Moreover, methods for preparing sustained-release drugs are known, and these can be applied to HGFs (Langer et al., J. Biomed. Mater. Res. (1981) 15, 167-277; Langer, Chem. Tech. (1982) 12, 98-105; U.S. Pat. No. 3,773,919; European Patent Application (EP) No. 58,481; Sidman et al., Biopolymers (1983) 22, 547-56; EP No. 133,988).

The lymphangiogenesis-promoting agents of the present invention can be administered to any mammal, including humans, rats, and dogs. These agents can be administered either orally or parenterally, but are preferably administered parenterally. Specifically, the agents can be administered to patients percutaneously or by injection. For example, injections can be locally administered by intravenous injection, intramuscular injection, or subcutaneous injection; however, they are preferably injected locally, particularly intramuscularly, in and around the areas where promotion of lymphangiogenesis is desired. Furthermore, the method of administration can be appropriately selected according to the age and symptoms of the patient

When an active ingredient is a protein, a single dose effective to promote lymphangiogenesis or to prevent or treat lymphedema can be selected from between 0.0001 to 10 mg per kg of body weight. Alternatively, when administered to human patients, the dose can be selected from between 0.001 to 100 mg/body, and the single dose preferably includes about 0.01 to 5 mg/body of HGF protein. However, the doses of the lymphangiogenesis-promoting agents of the present invention are not limited to these doses.

The present invention also relates to lymphangiogenesis-promoting agents comprising as active ingredients nucleic acids (hereinafter sometimes referred to as “the HGF genes”) encoding HGFs or proteins functionally equivalent thereto. The terms “HGF” and “protein functionally equivalent to an HGF” of the present invention are as described above. Also in this context, an HGF is preferably a human HGF.

Nucleic acids encoding HGFs of the present invention include cDNAs, genomic DNAs, and chemically synthesized DNAs. Moreover, nucleic acids encoding HGFs may include arbitrary sequences based on degeneracy of the genetic code, as long as they encode HGFs. Furthermore, nucleic acids encoding HGFs include, in addition to the DNAs described above, derivatives thereof and artificially modified nucleic acids. Such artificially modified nucleic acids include, for example, DNAs in which their sugar chain structures have been modified, cDNAs, genomic DNAs, chemically synthesized DNAs, and derivatives thereof, but are not limited thereto.

Genomic DNAs and cDNAs can be prepared by conventional methods known to those skilled in the art. Genomic DNAs can be prepared, for example, by extracting genomic

DNAs from human-derived cells, preparing genomic libraries (plasmids, phages, cosmids, BAC, PAC, and the like can be used as vectors), developing them, and carrying out colony or plaque hybridization using probes prepared based on nucleic acids encoding HGFs (for example, the nucleic acid of SEQ ID NO: 1). Alternatively, the DNAs can also be prepared by preparing primers specific to HGF-encoding DNAs and performing PCR using the primers. In addition, cDNAs can be prepared, for example, by synthesizing cDNAs based on mRNAs extracted from human-derived cells, preparing cDNA libraries in which the cDNAs are inserted into vectors such as λZAP, developing them, and carrying out colony or plaque hybridization as described above, or performing PCR.

Nucleic acids encoding HGFs or proteins functionally equivalent thereto that are comprised as active ingredients in the lymphangiogenesis-promoting agents of the present invention may be naked nucleic acids, or may be included in viral envelopes, liposomes or the like. The term “naked nucleic acid” refers to a nucleic acid in a state where the nucleic acid molecule is not present in an inclusion body, such as a viral envelope or liposome, but is present in an aqueous solution in a naked form without being coated.

Nucleic acids encoding HGFs or proteins functionally equivalent thereto are preferably incorporated into nucleic acid vectors. Any nucleic acid vector can be used in the present invention, as long as the nucleic acid encoding an HGF of the present invention that is inserted into the vector can be expressed in mammals. Such vectors include, for example, plasmids; viral vectors such as retroviral vectors, adenoviral vectors, adeno-associated virus vectors, vaccinia virus vectors, lentiviral vectors, herpes virus vectors, alphavirus vectors, EB virus vectors, papilloma virus vectors, and foamy virus vectors; and non-viral vectors (Niitsu Y. et al., Molecular Medicine 35: 1385-1395 (1998)), but are not limited thereto. Vectors in the present invention are suitable for use as vectors for gene therapy.

Those skilled in the art can appropriately design and use desired vectors. Vectors used in the present invention may include polynucleotide sequences that allow more efficient expression of HGFs, such as a transcription initiation region and transcription termination region that function in expression hosts, in addition to an arbitrary polynucleotide to be introduced.

Preferred forms of nucleic acids encoding HGFs used in the present invention include, for example, a nucleic acid encoding an HGF that has been inserted into a plasmid vector, such as pVAX1HGF/MGB1 described in SEQ ID NO: 5 or pcDNA3.1(-)HGF described in SEQ ID NO: 6.

Nucleic acids of the present invention that encode HGFs or proteins functionally equivalent to HGFs may be incorporated into vectors, such as cationic liposomes, ligand-DNA complexes, and gene guns. The nucleic acids may be, for example, in a form where they are packaged in HVJ-E vectors derived from Sendai virus envelopes (Kaneda, Y. et al., Mol. Ther. 6, 219-226 (2002)). Alternatively, the nucleic acids may be in a naked form.

In the present invention, the HGF genes may be used alone or in combination with other genes and/or other pharmaceutical agents as needed. In the present invention, the HGF gene and the genes used in combination as needed are incorporated into appropriate vectors that ensure the in vivo expression of the genes, and then administered to affected areas of patients.

When lymphangiogenesis-promoting agents comprising as active ingredients nucleic acids encoding HGFs or proteins functionally equivalent thereto are administered, those skilled in the art can readily select an adequate administration route and dose. However, the agents are preferably administered by injection to and/or around the areas where promotion of lymphangiogenesis is desired, in particular by intramuscular injection to the muscles in and/or around the areas. Other administration methods may be used, as long as HGFs or proteins functionally equivalent thereto can be expressed from the administered nucleic acids in and around the areas where lymphangiogenesis is desired to be promoted.

When an active ingredient is a nucleic acid described above, the single dose of the nucleic acid that is effective to promote lymphangiogenesis or to prevent or treat lymphedema can be selected, for example, from the range of 0.001 to 10 mg per kg body weight. Alternatively, when administered to human patients, the dose of the nucleic acid can be selected, for example, from the range of 0.001 to 50 mg/body, and the single dose of the nucleic acid is preferably about 0.5 to 50 mg/body. However, the dose is not limited to those described above.

In a preferred embodiment of the lymphangiogenesis-promoting agents comprising as active ingredients nucleic acids encoding HGFs or proteins functionally equivalent thereto, for example, when the agent is administered to a human, a plasmid including a human HGF cDNA (for example, pVAX1HGF/MGB1 described in SEQ ID NO: 5 or pcDNA3.1(-)HGF described in SEQ ID NO: 6) dissolved in a physiological buffer or pharmaceutically acceptable carrier is intramuscularly injected to the muscle in and/or around an area where lymphangiogenesis is desired to be promoted in a human patient at a single dose of 1 mg to 20 mg, preferably 1 mg to 10 mg, and more preferably 2 mg to 10 mg (for example, 3 mg to 6 mg, or 2 mg to 4 mg). The agent may be administered twice or more, preferably three times or more, at intervals of about 1 to 10 weeks, preferably about 1 to 8 weeks (for example, 2 to 6 weeks, or 1 to 4 weeks). When administered to humans, the agents may be administered, for example, according to the method described by Morishita R. et al., Hypertension 44(2):203-9 (2004), or an appropriately modified method thereof.

The lymphangiogenesis-promoting agents of the present invention can be used as pharmaceutical agents for preventing or treating lymphedema. Lymphedema refers to a condition where occlusion of lymphatic vessels causes abnormal congestion of tissue fluid, resulting in swelling, chronic inflammation, and/or fibrosis. Lymphedema that is a target for prevention or treatment by the lymphangiogenesis-promoting agents of the present invention include all conditions that have such symptoms as described above, regardless of their names. The lymphangiogenesis-promoting agents of the present invention are useful for preventing or treating diseases involving such symptoms.

In subjects at high risk for lymphedema (for example, patients whose lymph nodes have been extirpated along with malignant tumors by surgery), lymphedema can be prevented by administering the lymphangiogenesis-promoting agents of the present invention.

The present invention also relates to methods for preventing or treating lymphedema, which comprise the step of administering to subjects, HGFs, proteins or compounds functionally equivalent to HGFs, or nucleic acids encoding HGFs or proteins functionally equivalent thereto. Herein, HGFs are preferably human HGFs. The administration site may be any site where lymphangiogenesis is desired to be promoted. Lymphedema can also be treated, for example, by applying the above-described methods to patients with lymphedema. More specifically, lymphangiogenesis can be promoted by administering the lymphangiogenesis-promoting agents, as described above.

Those skilled in the art can administer human HGFs or the human HGF genes to affected areas of patients with lymphedema, appropriately considering the purpose. The administration to the affected areas of patients can be achieved by methods known to those skilled in the art.

In the present invention, the proteins, nucleic acids, and compounds described above in (1) to (4) may be administered alone or in combination with genes encoding other lymphangiogenic factors, or as compositions in combination with pharmaceutically acceptable carriers and/or additives. When administered in a form of composition, specific embodiments of genes encoding other lymphaniogenic factors, and pharmaceutically acceptable carriers and/or additives to be used in combination are as described above.

Affected areas to which the proteins, nucleic acids, and compounds described above in (1) to (4) are not particularly limited, so long as they are areas in which the symptoms of lymphedema have appeared or are predicted to appear. For example, local administration to the upper arm region can be considered for lymphedema of upper limb, which is often observed after breast cancer surgery and such; alternatively, local administration to the femoral region can be considered for lymphedema of lower limb, which is often observed after uterine cancer surgery and such.

The dose is as described above; however, it varies depending on the type of disease, patient's weight, age, sex, and symptoms, administration purpose, form of the gene to be introduced and such. Those skilled in the art can appropriately determine the dose.

The subjects to which compositions, including proteins, nucleic acids, or compounds of the present invention, are administered include any mammal, such as a monkey, dog, and cat, in addition to human.

Furthermore, in an embodiment of the present invention, the methods for promoting lymphangiogenesis also include methods in which activation of HGF receptors is induced and lymphangiogenesis is promoted through such activation.

HGF receptors are membrane proteins named c-met, and have a tyrosine kinase in their cytoplasmic domain. When an HGF binds to the HGF binding site in the extracellular domain of c-met, the above-mentioned cytoplasmic tyrosine kinase is activated and phosphorylates tyrosine residues (Y¹³⁴⁹ and Y¹³⁵⁶) in the c-met molecule. This triggers activation of the intracellular signaling pathway and then the HGF exerts its biological function. This mechanism is well-known in the art (see, for example, Graziani, A. et al., J. Biol. Chem. 266, 22087-22090 (1991); Graziani, A. et al., J. Biol. Chem. 268, 9165-9168 (1993); and Nakagami, H. et al., Hypertension, 37 [part2]: 581-586 (2001)). Thus, herein, the activation of an HGF receptor refers to the activation of tyrosine kinase in the c-met molecule.

In the present invention, lymphangiogenesis can be promoted by activating c-met expressed in lymphatic endothelial cells. The c-met activation is induced by reacting HGFs, or proteins or compounds functionally equivalent thereto with c-met on lymphatic endothelial cells. Thus, the lymphangiogenesis-promoting agents described above can also be utilized in this embodiment.

The present invention relates to methods of screening for compounds having lymphangiogenesis-promoting activity or compounds having an effect of preventing or treating lymphedema.

In an embodiment of the present invention, the screening methods are methods of screening for compounds having lymphangiogenesis-promoting activity, which comprise the following steps of (a) to (c):
(a) contacting test compounds with HGF receptors or proteins functionally equivalent thereto;
(b) detecting the binding of test compounds to HGF receptors or proteins functionally equivalent thereto; and
(c) selecting test compounds that bind to HGF receptors or proteins functionally equivalent thereto.

In the first embodiment, HGF receptors or proteins functionally equivalent thereto are first contacted with test compounds. The HGF receptors and proteins functionally equivalent thereto in the screening methods of the present invention include c-met and proteins functionally equivalent thereto. Specifically, such proteins include the following (i) to (iv):
(i) proteins having the amino acid sequence of SEQ ID NO: 4;
(ii) proteins encoded by nucleic acids that include the coding region of the nucleotide sequence of SEQ ID NO: 3;
(iii) proteins which have an amino acid sequence with a substitution, deletion, insertion, and/or addition of one or more amino acids in the amino acid sequence of SEQ ID NO: 4, and which are functionally equivalent to proteins having the amino acid sequence of SEQ ID NO: 4; and
(iv) proteins encoded by nucleic acids that hybridize under stringent conditions to nucleic acids having the nucleotide sequence of SEQ ID NO: 3, which are functionally equivalent to proteins having the amino acid sequence of SEQ ID NO: 4.

The “test compounds” in the methods of the present invention are not particularly limited, and include, for example, single compounds such as natural compounds, organic compounds, inorganic compounds, proteins, and peptides; as well as compound libraries, expression products of gene libraries, cell extracts, cell culture supernatants, products of fermentation microorganisms, marine organism extracts, plant extracts, prokaryotic cell extracts, unicellular eukaryote extracts, and animal cell extracts. If needed, the above test compounds can be appropriately labeled before use. Labels include, for example, radiolabels and fluorescent labels.

In the present invention, “contacting” is achieved by the procedure described below. For example, “contacting” can be achieved by adding a test compound to a culture medium of cells expressing an HGF receptor or a protein functionally equivalent thereto or an extract of cells expressing an HGF receptor or a protein functionally equivalent thereto. When the test compound is a protein, “contacting” can be achieved, for example, by introducing a vector carrying a DNA encoding the protein into cells expressing an HGF receptor or a protein functionally equivalent thereto, or by adding the vector to an extract of cells expressing an HGF receptor or a protein functionally equivalent thereto. Alternatively, for example, the two hybrid method with yeast, animal cells or the like can be used.

In the first embodiment, the binding between the test compounds and HGF receptors or proteins functionally equivalent thereto is subsequently detected. Detection or measurement of the binding between proteins can be carried out by using, for example, labels attached to the proteins. The types of labels include, fluorescent labels and radiolabels, for example. The binding can also be measured by known methods such as the yeast two hybrid method and the measurement method using BIACORE. In the present methods, the test compounds bound to the above-mentioned HGF receptors are then selected. The selected test compounds include compounds having lymphangiogenesis-promoting activity or compounds having an effect of preventing or treating lymphedema. In addition, the selected test compounds may be used as test compounds in the following screening.

Furthermore, in the second embodiment of the screening methods, the present invention provides methods of screening for compounds having lymphangiogenesis-promoting activity or compounds having an effect of preventing or treating lymphedema, which comprise the following steps (a) to (c):
(a) contacting test compounds with cells expressing HGF receptors or proteins functionally equivalent thereto;
(b) measuring the growth capacity or migratory activity of the cells, or phosphorylation of signaling molecules;
(c) selecting test compounds that increase the growth capacity or migratory activity of the cells, or that cause phosphorylation of signaling molecules, as compared to when the test compounds are not contacted.

In the second embodiment, test compounds are first contacted with cells expressing

HGF receptors or proteins functionally equivalent thereto. The “cells expressing HGF receptors or proteins functionally equivalent thereto” include isolated cells expressing HGF receptors or proteins functionally equivalent thereto and transformed cells expressing recombinant HGF receptors, but are not limited thereto. These cells include, for example, lymphatic endothelial cells derived from thoracic duct. HGF receptors or proteins functionally equivalent thereto used in the instant methods include the HGF receptors described above and proteins functionally equivalent thereto.

In the second embodiment, then, cells expressing HGF receptors or proteins functionally equivalent thereto are measured for their growth capacity or migratory activity, or phosphorylation of signaling molecules such as MAPK, Akt, c-met, and Ras, which are known to be phosphorylated by the c-met signaling.

Those skilled in the art can readily measure the degree of an increase in growth capacity and migratory activity, and the phosphorylation of MAPK or Akt, for example, by the methods described below in Examples.

In the screening methods of the present invention, the final step is selection of compounds that increase the growth capacity or migratory activity of the cells expressing HGF receptors or proteins functionally equivalent thereto, or that cause phosphorylation of molecules such as MAPK, Akt, c-met, and Ras, which are known to be phosphorylated by the c-met signaling, as compared to when the test compounds are not contacted. The compounds thus selected can be used as active ingredients of the lymphangiogenesis-promoting agents of the present invention or the pharmaceutical agents of the present invention for preventing or treating lymphedema.

All prior art documents cited herein are incorporated herein by reference.

### EXAMPLES

Herein below, the present invention will be specifically described with reference to Examples, but is not to be construed as being limited thereto.

### Example 1

### Effect of HGFs on Primary Cultured Lymphatic Endothelial Cells Derived from Canine Thoracic Duct

(1) Preparation of Primary Cultured Lymphatic Endothelial Cells Derived from the Canine Thoracic Duct

Primary cultured lymphatic endothelial cells derived from the canine thoracic duct were prepared by a known method (Microcirculation 6, 75-78 (1999)). Adult mongrel dogs (including both male and female; 6 to 12 kg) were sacrificed by bleeding from the femoral artery under anesthesia. Then, the thoracic ducts (10 to 15 cm) were isolated and placed in cold (4° C.) Hanks' balanced salt solution (HBSS). Connective tissues and adipocytes were removed. Branches of the thoracic ducts were ligated with sterilized silk thread, and the lymphatic vessels were washed with cold HBSS. Next, the thoracic ducts were incubated in a collagenase solution (250 U/ml HBSS) at 37° C. for 10 minutes. The ducts were washed with MEM containing 10% fetal bovine serum (FBS) and the cells were collected. After collagenase was removed by centrifugation, the cells were suspended in complete MEM supplemented with 20% FBS and an antibiotic, and then cultured in 35-mm dishes coated with type I collagen. The cells were cultured under a wet condition with 5% CO₂ at 37° C. When the cells reached confluence, they were detached with trypsin/EDTA and collected. Lymphatic endothelial cells were subcloned to prepare lymphatic endothelial cell clones. The clonal cells were able to be passaged at least 10 or more times.

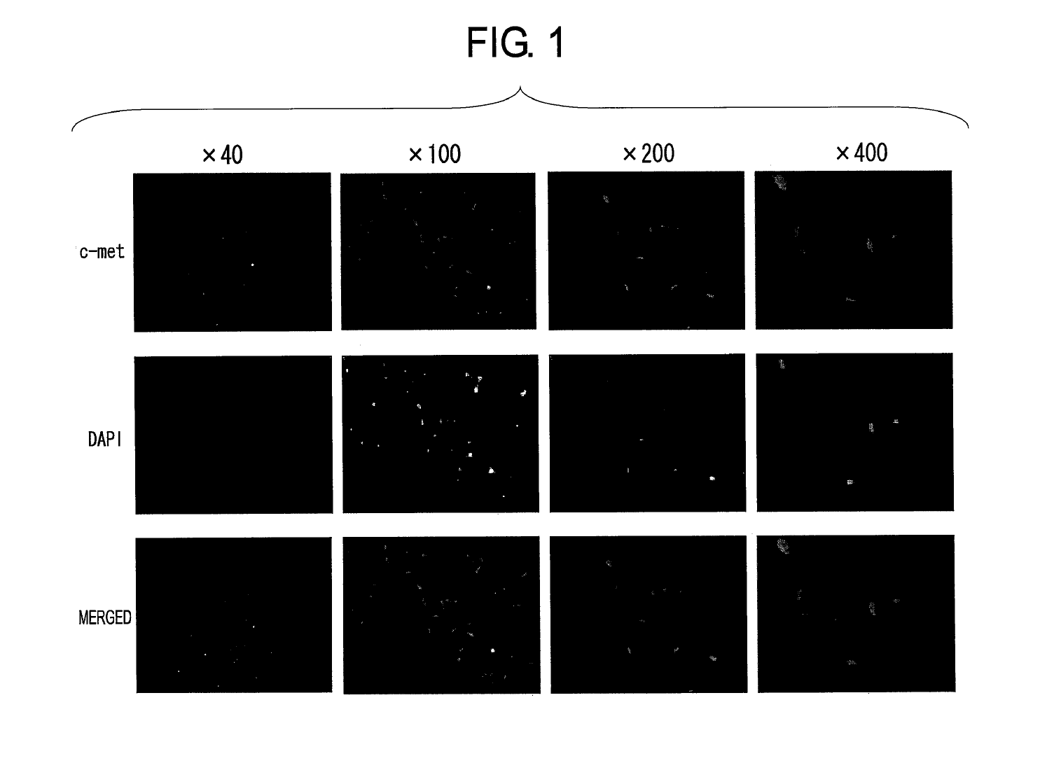

The lymphatic endothelial cells thus obtained were confirmed to be immunostained with different types of lymphatic endothelial cell-specific antibodies (including anti-VEGFR-3 antibody and anti-Prox1 antibody). Furthermore, the expression of c-met, an HGF receptor, in these cells was confirmed by immunostaining. This novel finding is attributable to the present invention (FIG. 1).

### 2) Enhancement of Growth Capacity by a Recombinant Human HGF

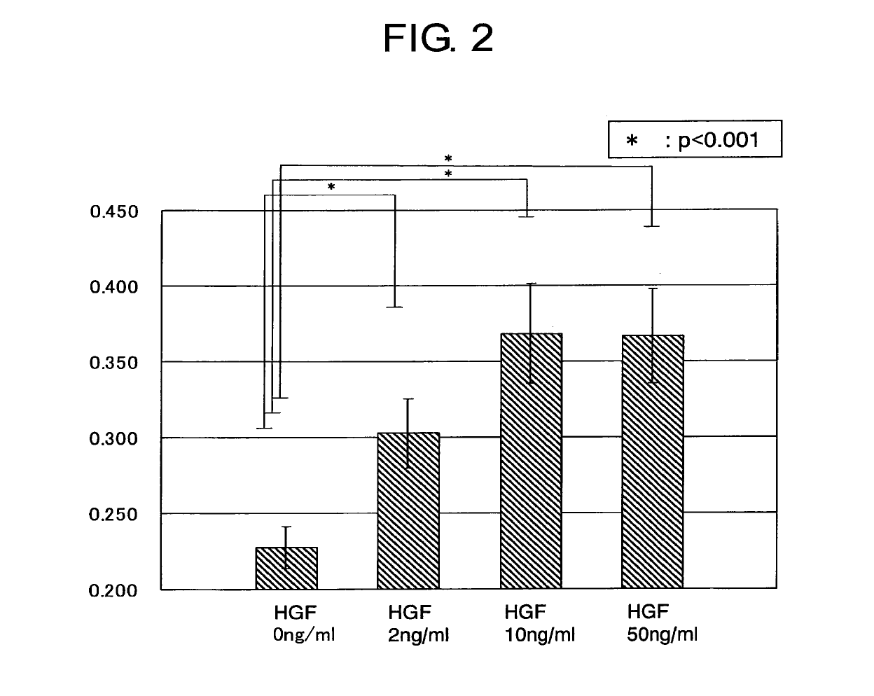

A recombinant human HGF was added at a concentration of 0, 2, 10, or 50 ng/ml to medium containing the lymphatic endothelial cells derived from the canine thoracic duct (70% or more confluent in 96-well plates) prepared above in (1). After three days, the MTS assay was carried out using the CellTiter 96 One Solution Reagent (Promega). The assay was independently performed on nine wells for each concentration. The results are shown in Table 1 and FIG. 2.

TABLE 1

| | | | | HGF |
|---|---|---|---|---|
| | HGF 0 ng/ml | HGF 2 ng/ml | HGF 10 ng/ml | 50 ng/ml |
| No 1 | 0.22 | 0.257 | 0.339 | 0.285 |
| No 2 | 0.203 | 0.26 | 0.291 | 0.299 |
| No 3 | 0.203 | 0.279 | 0.344 | 0.341 |
| No 4 | 0.183 | 0.248 | 0.251 | 0.25 |
| No 5 | 0.208 | 0.262 | 0.3 | 0.301 |
| No 6 | 0.193 | 0.247 | 0.338 | 0.406 |
| No 7 | 0.26 | 0.367 | 0.403 | 0.477 |
| No 8 | 0.288 | 0.417 | 0.499 | 0.503 |
| No 9 | 0.289 | 0.387 | 0.549 | 0.437 |
| Mean | 0.227 | 0.303 | 0.368 | 0.367 |
| Standard | 0.041 | 0.068 | 0.099 | 0.092 |
| deviation (SD) | | | | |
| Standard | 0.014 | 0.023 | 0.033 | 0.031 |
| error (SE) | | | | |

Even in the presence of 2 ng/ml of HGF, the measured values significantly increased. These results suggest that the HGF strongly promotes the growth of lymphatic endothelial cells.

### (3) Enhancement of Migratory Activity by a Recombinant Human HGF

### (i) Enhancement of Migratory Activity by a Recombinant HGF

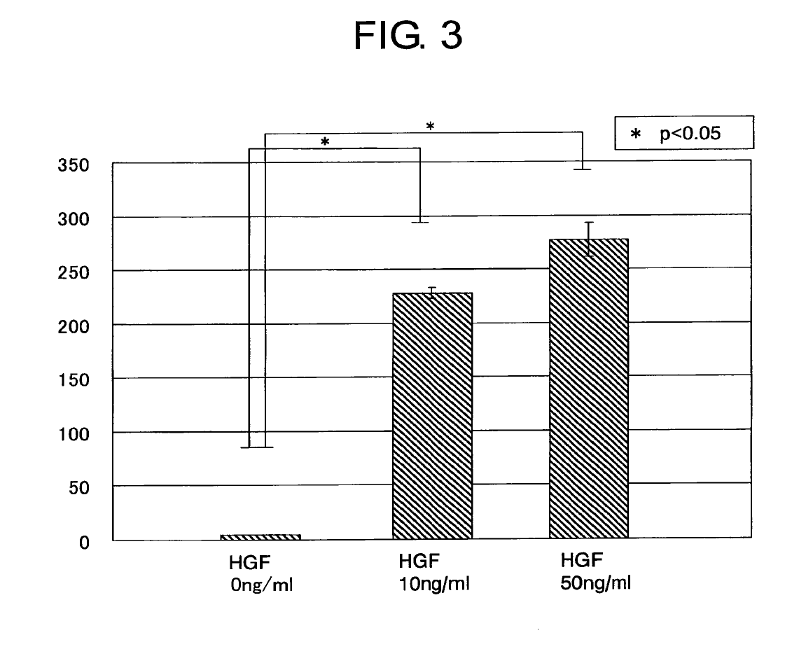

The migratory activity of cells was measured by a previously reported method using the Boyden chamber (Arterioscler Thromb Vasc Biol. 22:108-114 (2002)). A polyvinylpyrrolidone (PVP)-free polycarbonate membrane with a pore size of 8 μm (Neuro Probe Inc., Gaithersburg, Md.) was coated with 0.1% gelatin and excess gelatin was removed using phosphate buffered saline (PBS). The above-described membrane was placed onto the lower chamber of the Boyden chamber containing 28 μl of EBM2 medium supplemented with 1% FBS, and 50 μl of medium containing 10⁶ of the lymphatic endothelial cells derived from the canine thoracic duct prepared above in (1) was added to the upper chamber. A recombinant human HGF was added to the medium at a concentration of 0, 10, or 50 ng/ml. The cells were cultured under a wet condition with 5% CO₂ at 37° C. for four hours. The membrane was removed, and the cells on the upper surface of the membrane were detached. The cells on the lower surface of the membrane were stained with the Diff-Quick (Sysmex, Hyogo, Japan) and counted. This assay was independently performed in three wells for each HGF concentration. The results are shown in Table 2 and FIG. 3

TABLE 2

| | HGF (—) | HGF 10 ng/ml | HGF 50 ng/ml |
|---|---|---|---|
| No 1 | 3 | 224 | 271 |
| No 2 | 4 | 238 | 307 |
| No 3 | 6 | 222 | 254 |
| Mean | 4.33 | 228.00 | 277.33 |
| SD | 1.53 | 8.72 | 27.06 |
| SE | 0.0882 | 5.033 | 15.624 |

Table 2 shown above and FIG. 3 suggest that even in the presence of 10 ng/ml of HGF, the measured values significantly increases and thus the HGF promotes migration of lymphatic endothelial cells.

The above results suggest that HGFs promote lymphangiogenesis by promoting the growth and migration of lymphatic endothelial cells.

### (ii) Enhancement of Migratory Activity by a Paracrine Recombinant HGF

An expression plasmid for human HGF cDNA, pVAX1HGF/MGB1 (SEQ ID NO: 5), was packaged in the HVJ-E vector by a previously reported method (Kaneda, Y. et al., Mol. Ther. 6, 219-226 (2002)).

BHK cells were cultured until they reached confluence in 100-mm dishes. 50 HAU of the vector containing the plasmid was added to the culture medium of the BHK cells and then introduced into the cells. After 24 hours of culture, the cells were transferred into inserts for 24-well plates and cultured until they were almost confluent.

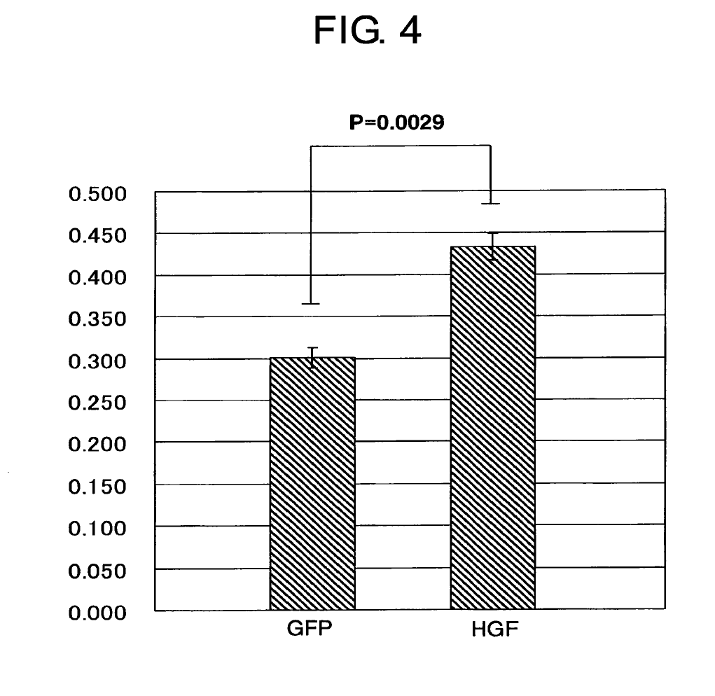

Meanwhile, the lymphatic endothelial cells derived from the canine thoracic duct descried above in (1) were cultured until 70% or more confluent in 24-well plates, and the inserts containing the above-described BHK cells introduced with the expression plasmid for human HGF cDNA pVAX1HGF/MGB1 were placed in the wells. After 48 hours of culture, in the same way as described above, the cells were subjected to the MTS assay. The assay was independently performed on six wells for each sample. The cells treated in the same way using a GFP expression plasmid instead of the expression plasmid for human HGF cDNA pVAX1HGF/MGB1, were used as a negative control. The results are shown in Table 3 and FIG. 4.

TABLE 3

| | GFP | HGF |
|---|---|---|
| No 1 | 0.260 | 0.497 |
| No 2 | 0.320 | 0.451 |
| No 3 | 0.338 | 0.397 |
| No 4 | 0.272 | 0.414 |
| No 5 | 0.306 | 0.440 |
| No 6 | 0.307 | 0.400 |
| Mean | 0.301 | 0.433 |
| SD | 0.029 | 0.038 |
| SE | 0.012 | 0.016 |

When the cells were co-cultured with the cells introduced with the HGF cDNA, the measured values in the MTS assay were significantly increased as compared to the negative control. These results demonstrate that the introduction of the HGF gene is effective to promote the growth of lymphatic endothelial cells.

To further confirm the promotion of lymphatic endothelial cell growth by the HGF, c-fos promoter activity was measured by a previously reported method (Hypertension 37(2); 581-586 (2001)).

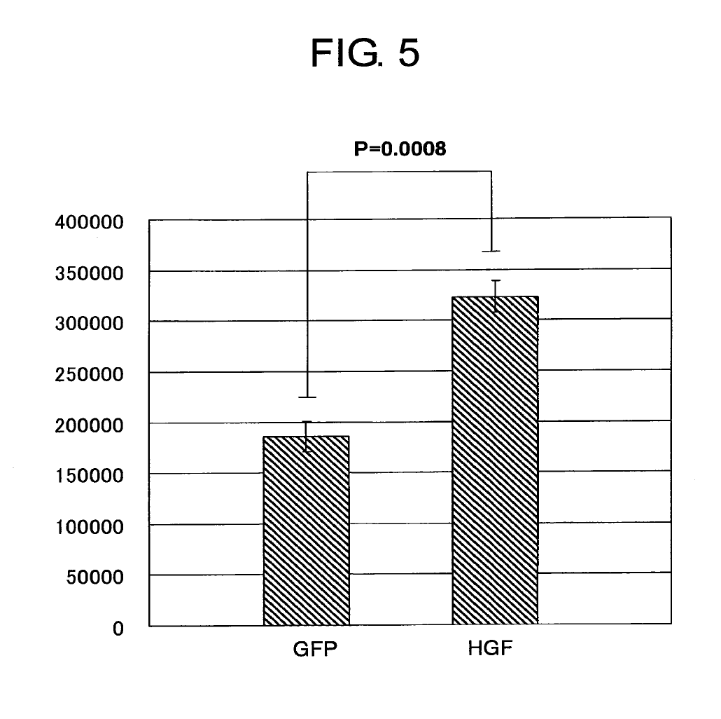

The assay was independently performed on six wells for each sample. The cells treated using a GFP expression plasmid in the same way as described above, were used as a negative control. The results are shown in Table 4 and FIG. 5.

TABLE 4

| | GFP | HGF |
|---|---|---|
| No 1 | 231101 | 317234 |
| No 2 | 210645 | 344423 |
| No 3 | 143101 | 303133 |
| No 4 | 141224 | 319125 |
| No 5 | 195948 | 384934 |
| No 6 | 189940 | 270410 |
| Mean | 185326.50 | 323209.83 |
| SD | 36327.55 | 38738.02 |
| SE | 14830.66 | 15814.73 |

The c-fos promoter activity is known to be positively correlated with the cell growth capacity. Thus, given the fact that the HGF increases the c-fos promoter activity, it is apparent that the introduction of the HGF gene is effective to promote the lymphatic endothelial cell growth.

(5) Comparison of Growth Promoting Effects Between a Human HGF cDNA And VEGF cDNA in an Autocrine System

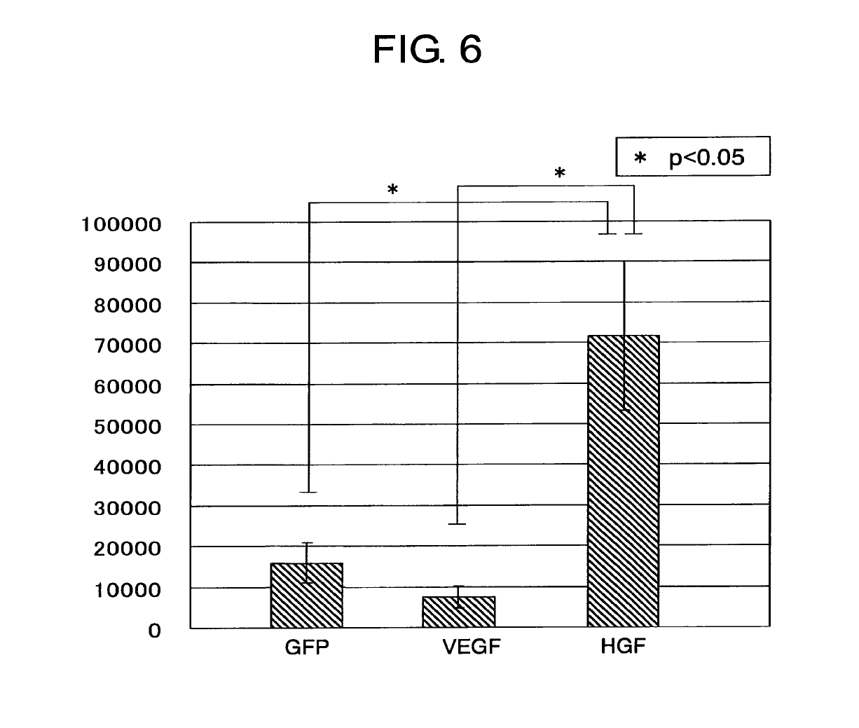

Using Lipofectamine Plus (GIBCO-BRL), an expression plasmid for a naked human HGF cDNA, pVAX1HGF/MGB1, and a c-fos luciferase reporter gene plasmid (p2FTL) for c-fos promoter assay were introduced into the above-described lymphatic endothelial cells cultured until subconfluent in 6-well plates (J. Hypertens. 16: 993-1000 (1998)). After 24 hours of culture, the cells were cultured in serum-free culture medium for 24 hours. The luciferase activity was then determined by a conventional method, and found to be increased. Next, using the human VEGF cDNA instead of the human HGF cDNA, the same experiments as described above in (4) were carried out to compare the effects of human HGF cDNA and VEGF cDNA on the c-fos promoter activity. The assay was independently performed on four wells for each sample. The results are shown in Table 5 and FIG. 6.

TABLE 5

| | GFP | VEGF | HGF |
|---|---|---|---|
| No 1 | 28135 | 9213 | 120035 |
| No 2 | 19431 | 14273 | 80231 |
| No 3 | 6789 | 3728 | 46335 |
| No 4 | 9085 | 2936 | 39688 |
| Mean | 15860.00 | 7537.50 | 71572.25 |
| SD | 9859.20 | 5287.10 | 36865.23 |
| SE | 4929.599 | 2643.551 | 18432.617 |

These results suggest that introduction of the expression plasmid for HGF cDNA significantly increases the c-fos promoter activity through the autocrine of HGF, while the expression plasmid for VEGF cDNA does not increase the c-fos promoter activity and thus does not promote the lymphatic endothelial cell growth.

The results of experiments using the primary cell culture system described above suggest that lymphangiogenesis is promoted by HGFs, that lymphangiogenesis can be promoted by introducing the HGF gene, and that the introduction of an expression plasmid for a naked HGF cDNA, namely the introduction of a nucleic acid encoding an HGF, is effective for the promotion.

Furthermore, lymphatic endothelial cells isolated from adult dogs were used in the present Example, and thus HGFs were demonstrated to have the above-described activity on lymphatic endothelial cells of adult animals. This suggests that HGFs are useful as therapeutic agents for lymphedema.

Moreover, in order to confirm that the lymphangiogenesis-promoting activity of human HGF was not specific to canine lymphatic endothelial cells, the growth-promoting activity of HGF on primary cultured aortic and venous endothelial cells isolated from adult mongrel dogs in the same way as described above was investigated. As a result, the human HGF was found to have growth-promoting activity on these cells. Furthermore, this growth-promoting activity was comparable to that on equivalent human cells (data not shown). Consequently, human HGFs also act on canine vascular endothelial cells in the same manner as on human vascular endothelial cells. These findings suggest that the lymphangiogenesis-promoting activity of human HGFs is not specific to canine lymphatic endothelial cells.

### (6) Confirmation of HGF Activity on Human Lymphatic Endothelial Cells

It was postulated that human lymphatic endothelial cells would give the same result as that obtained using the lymphatic endothelial cells isolated form adult dogs. To confirm this prediction, the effect of HGF on the growth and migratory capacities of human lymphatic endothelial cells was assayed (AngioBio Co. (Del Mar, Calif.)).

The cells at passage 5 to 8 were used in the experiments. By immunostaining, it was confirmed that von Willebrand factor, VEGF receptor-3, Prox1, and c-Met, which are lymphatic endothelial cell markers, were also expressed in the cells (data not shown). The MTS assay and migration assay were carried out in the same way as the experiments using canine lymphatic endothelial cells.

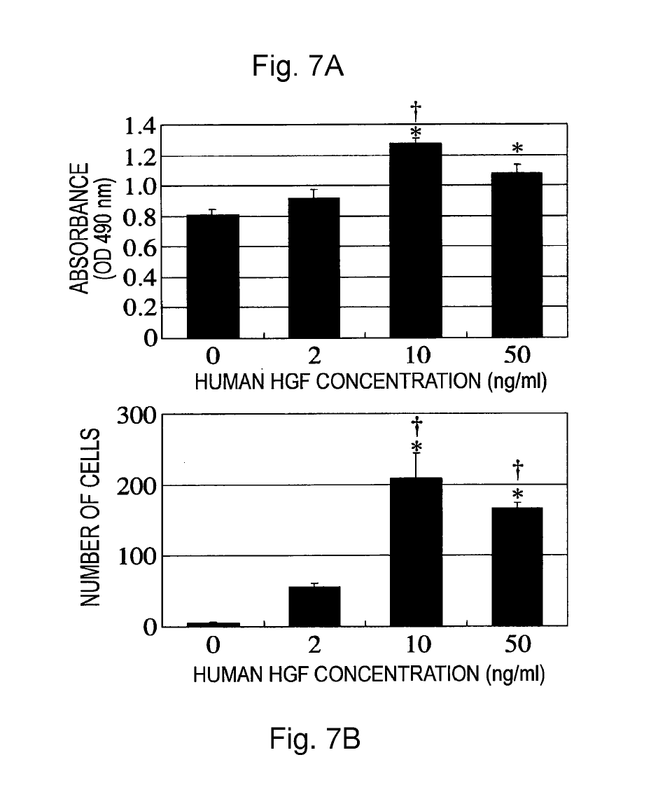

The results of MTS assay and migration assay are shown in FIGS. 7A and 7B, respectively. Both cell growth capacity and migratory capacity were increased by adding a recombinant human HGF. They were significantly increased at 10 ng/ml or higher concentrations as compared to the cells in the absence of HGF.

The results confirm the initial prediction that an HGF would promote lymphangiogenesis of human lymphatic endothelial cells as well as the lymphatic endothelial cells from adult dogs described above in (1) to (5).

The above results suggest that HGFs have lymphangiogenesis-promoting activity not only on dogs but also on other mammals (including humans).

### Example 2

### Effect of HGFs on Rat Models of Lymphedema

Based on the findings described above, the effect of an HGF on lymphedema was tested using rat models to demonstrate the in vivo effect.

### (1) Preparation of Rat Models of Lymphedema

Rat models of Lymphedema were prepared according to Slavin S A et al. (Anals of Surgery 229, 421-427 (1999)). To confirm the presence of lymphedema in the tail of rat models, physiological saline containing 0.5% Patent Blue dye was injected at a position several centimeters distant from the base of tail immediately before surgery. The tail region was dissected on the day following the surgery, and the blue dye was observed in the lymphatic vessels (data not shown). Furthermore, the base of tail was evidently thicker as compared to control rats, and thus the above rats were demonstrated to be useful as lymphedema models.

(2) Lymphedema-improving Effect of an Expression Plasmid for HGF cDNA on Rat Models of Lymphedema

The effect of introduction of the human HGF gene on lymphedema was investigated using the rat models prepared as described above.

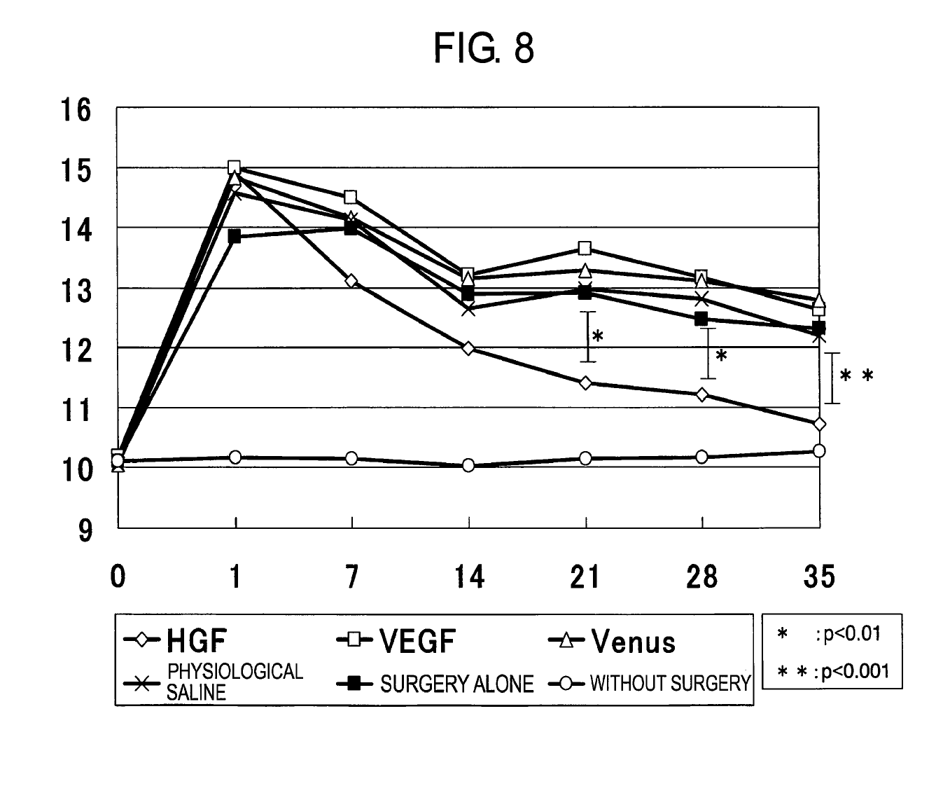

200 μg (/100 μl) of an expression plasmid for a naked human HGF cDNA, pVAX1HGF/MGB1, and 200 μg (/100 μl) of an expression plasmid for a naked VEGF cDNA, and as a control, 200 μg (/100 μl) of a naked GFP expression plasmid (Venus plasmid) were intramuscularly administered one, seven, and 14 days after surgery. In the group with surgery alone, only surgery was carried out without injection. In the group without surgery, no surgery was carried out. In the physiological saline group, 100 μl of physiological saline was intramuscularly injected one, seven, and 14 days after surgery. The tail thickness in each group was measured every seven days up to day 35 after surgery. Five rats in each group were tested and the mean was determined, which is shown in FIG. 8.

In all groups, the tail thickness transiently increased after surgery. However, the tail thickness was more rapidly decreased and the degree was greater only in the group introduced with HGF cDNA as compared to the other groups. This difference was significant after day 21. Thus, lymphedema was found to be improved by introducing the naked HGF cDNA plasmid.

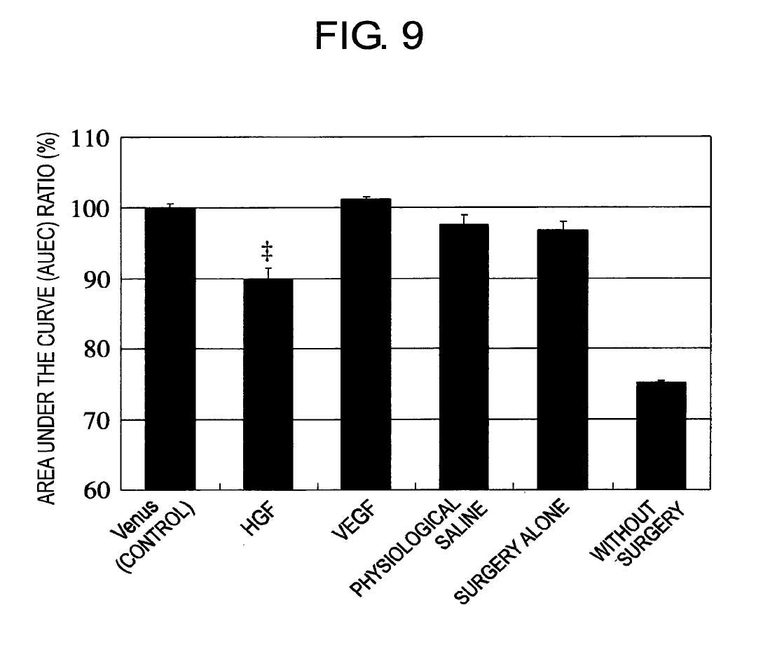

The areas under the curves shown in FIG. 8 were determined. The result is shown in FIG. 9. There was no difference between rats introduced with the VEGF gene and rats in the control Venus group; however, the tail thickness was found to be significantly decreased in rats introduced with the HGF gene. This decrease in the thickness of tail affected with lymphedema implies the relief or cure of lymphedema. Thus, lymphedema was clearly demonstrated to be relieved or cured by introducing a nucleic acid encoding an HGF (the HGF gene).

Tissue samples were collected from the above-described surgical site in the rat tails on days 4, 10, and 17 after surgery, and human HGF mRNA level was determined by real-time RT-PCR using a conventional method. As a result, human HGF expression was confirmed up to day 17 after surgery only in the group introduced with the human HGF gene (data not shown).

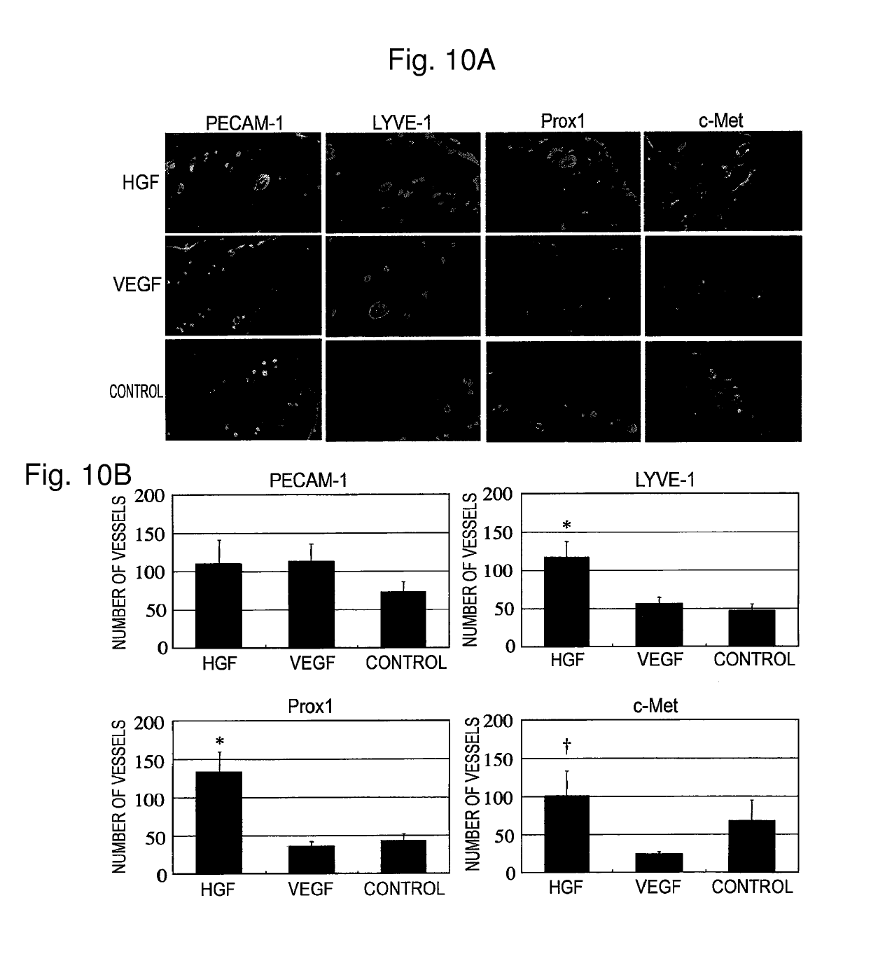

Furthermore, in the rats introduced with the HGF gene, rats introduced with the VEGF gene, and control rats injected with physiological saline, expression of endothelial cell marker (PECAM-1), lymphatic endothelial cell markers (LYVE-1 and Prox1), and c-met was detected by immunostaining at the injection sites on day 35 after surgery. Typical staining images are shown in FIG. 10 (FIG. 10A). In addition, the expression level of each marker was determined by counting the number of immunostaining-positive vessels in microscopic fields randomly selected using a known method (Yoon YS, et al., J. Clin. Invest. 111: 717-725 (2003)) (FIG. 10B). This result showed that there was no difference in the number of vessels positive for an endothelial cell marker, PECAM-1, in both rats introduced with the HGF gene and rats introduced with the VEGF gene as compared to the control. On the other hand, the numbers of vessels positive for lymphatic endothelial cell markers, LYVE-1 and Prox1, were both significantly increased in the rats introduced with the HGF gene, while there was no difference between the control and rats introduced with the VEGF gene. Furthermore, the number of vessels positive for c-met also tended to increase only in the rats introduced with the HGF gene as compared to the control. These results confirm that the HGF gene promotes lymphangiogenesis but the VEGF gene does not.

The results obtained with the rat models of lymphedema demonstrate that when a nucleic acid encoding an HGF is injected and expressed near sites affected with lymphedema, lymphatic vessels are newly generated around the injection sites. The rat models can reflect any type of lymphedema. Thus, the findings obtained herein with the rat models, that the symptom of lymphedema is relieved or cured in vivo by administering the HGF gene, suggest that HGFs and their genes are useful as therapeutic agents for lymphedema in mammals including humans.

### Example 3

### Confirmation of HGF Signaling in Lymphatic Endothelial Cells

To confirm the above-described mechanism of action of HGFs, it was demonstrated that phosphorylation of MAPK and Akt, which is known to be induced by HGFs in vascular endothelial cells, was also induced in lymphatic endothelial cells by HGF stimulation. The phosphorylation of MAPK and Akt is known to be essential for vascular endothelial cell growth promoted by HGFs (Nakagami H., Hypertension 37[part 2]: 581-586 (2001)).

The culture medium of the above-described lymphatic endothelial cells derived from canine thoracic duct was changed with MEM containing 0.5% FCS or FCS-free MEM 12 hours or more before addition of an HGF. A recombinant human HGF was added at a concentration of 100 ng/ml, and after 0 to 15 minutes the medium was removed and the cells were lysed with lysis buffer (50 mM Tris-Cl, 2.5 mM EGTA, 1 mM EDTA, 10 nM NaF, 1% deoxycorticosterone, 1% Triton X-100, 1 nM PMSF, and 2 mM sodium vanadate (pH 7.5)). The genome molecules were disrupted by sonication. Samples containing 20 μg proteins were subjected to 10% SDS-PAGE according to a conventional method. After transfer to nitrocellulose membrane, Western blotting was carried out using an anti-MAPK/ERK antibody, antibody specific to phosphorylated MAPK/ERK (phosphospecific; Tyr705 or Ser727), anti-Akt antibody, and antibody specific to phosphorylated Akt. The used primary antibodies were available from Cell Signaling Technology and others. Detection was achieved using the ECL kit (Amersham).

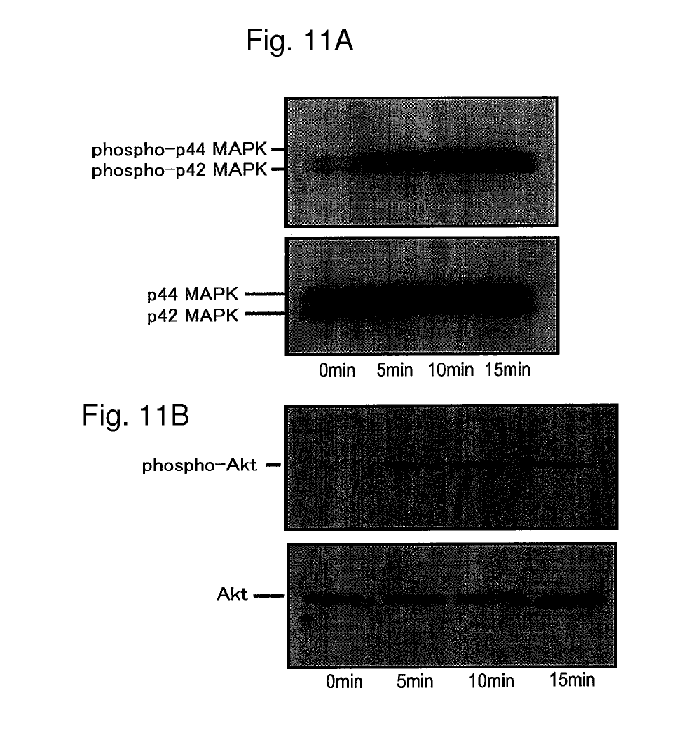

The results are shown in FIG. 11. FIG. 11A shows a result of Western blotting for MAPK. The result demonstrate that the phosphorylation of both p44 and p42 MAPKs is enhanced within five minutes after HGF stimulation. The result of Akt is shown in FIG. 11B. Likewise, the phosphorylation of Akt is enhanced within five minutes after HGF stimulation. Each bottom panel depicts detection of p44 and p42 MAPKs, or Akt as an internal control.

The phosphorylation of MAPK and Akt was also enhanced in lymphatic endothelial cells in response to HGF stimulation, as described above. This suggests that, in lymphatic endothelial cells, HGFs also induce, via c-met, the same phosphorylation cascade as in vascular endothelial cells. Thus, the phosphorylation of MAPK and Akt is presumed to be an essential signal for lymphatic endothelial cell growth.

Furthermore, the lymphatic endothelial cell growth-promoting activity of HGFs was found to be reduced by the MEK inhibitors U0126 (50 μM) and PD9805 (30 μM) and the PI3 kinase inhibitors Ly294002 (50 μM) and wortmannin (100 nM) (data not shown). This finding also suggests that the lymphangiogenic activity of HGFs is based on induction via c-met of the same signal cascade as in vascular endothelial cells by HGFs.

### INDUSTRIAL APPLICABILITY

The present invention provides novel lymphangiogenesis-promoting agents. The lymphangiogenesis-promoting agents provided by the present invention comprise human HGFs as active ingredients.

VEGF-C is a known peptidic factor that promotes lymphangiogenesis. However, other VEGF members belonging to the VEGF family have no lymphangiogenesis-promoting activity. Only VEGF-C has been known to have lymphangiogenesis-promoting activity.

Meanwhile, HGFs, which were discovered herein to have lymphangiogenesis-promoting activity, are known as angiogenic factors, like VEGF. Since VEGF has no lymphangiogenesis-promoting activity, those skilled in the art have presumed that HGFs also have no lymphangiogenesis-promoting activity. Accordingly, the findings of the present invention, that HGFs have lymphangiogenic activity, is considered as a remarkable fact.

In addition, VEGF-C may induce edema via cross-linking with VEGFR2; however, HGFs do not have such a risk. This is also a remarkable feature of the lymphangiogenesis-promoting agents of the present invention. The lymphangiogenesis-promoting agents are effective for preventing or treating lymphedema.

Furthermore, HGFs activate the growth and migration of lymphatic endothelial cells isolated not from fetal or neonatal systems but from adult animals, and thereby promote lymphangiogenesis. Most of patients suffering from lymphedema after surgical removal of cancer tissues and/or lymph nodes for cancer treatment are adult. Thus, the agents of the present invention are particularly useful as therapeutic agents for adult suffering from lymphedema after surgical removal of cancer tissues and/or lymph nodes for cancer treatment.

1.-7. (canceled)

8. A method for promoting lymphangiogenesis, which comprises the step of administering to a subject an HGF or a protein or compound functionally equivalent to an HGF.

9. The method of claim 8, wherein the HGF or protein functionally equivalent to an HGF is any one of the following (a) to (d):
(a) a protein comprising the amino acid sequence of SEQ ID NO: 2;
(b) a protein encoded by a nucleic acid comprising the coding region of the nucleotide sequence of SEQ ID NO: 1;
(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2; and
(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2.

10. A method for promoting lymphangiogenesis, which comprises the step of administering to a subject a nucleic acid encoding an HGF or a protein functionally equivalent to an HGF.

11. The method of claim 10, wherein the nucleic acid encoding an HGF or a protein functionally equivalent to an HGF is a nucleic acid encoding any one of the following (a) to (d):
(a) a protein comprising the amino acid sequence of SEQ ID NO: 2;
(b) a protein encoded by a nucleic acid comprising the coding region of the nucleotide sequence of SEQ ID NO: 1;
(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2; and
(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2.

12. The method of claim 10, wherein the nucleic acid has been inserted into a mammalian expression vector.

13. The method of claim 10, wherein the nucleic acid is a naked nucleic acid.

14. A method for inducing activation of an HGF receptor and promoting lymphangiogenesis through the activation.

15. A method for preventing or treating lymphedema, which comprises the step of administering to a subject an HGF or a protein or compound functionally equivalent to an HGF.

16. The method of claim 15, wherein the HGF or protein functionally equivalent to an HGF is any one of the following (a) to (d):
(a) a protein comprising the amino acid sequence of SEQ ID NO: 2;
(b) a protein encoded by a nucleic acid comprising the coding region of the nucleotide sequence of SEQ ID NO: 1;
(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2; and
(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2.

17. A method for preventing or treating lymphedema, which comprises the step of administering to a subject a nucleic acid encoding an HGF or a protein functionally equivalent to an HGF.

18. The method of claim 17, wherein the nucleic acid encoding an HGF or a protein functionally equivalent to an HGF is a nucleic acid encoding any one of the following (a) to (d):
(a) a protein comprising the amino acid sequence of SEQ ID NO: 2;
(b) a protein encoded by a nucleic acid comprising the coding region of the nucleotide sequence of SEQ ID NO: 1;
(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2; and
(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2.

19. The method of claim 17, wherein the nucleic acid has been inserted into a mammalian expression vector.

20. The method of claim 17, wherein the nucleic acid is a naked nucleic acid.

21.-26. (canceled)

27. A method of screening for a compound having lymphangiogenesis-promoting activity or a compound having an effect of preventing or treating lymphedema, wherein the method comprises the following steps:
(a) contacting a test compound with an HGF receptor or a protein functionally equivalent to an HGF receptor;
(b) detecting the binding between the protein and test compound; and
(c) selecting a test compound that binds to the protein.

28. The method of claim 27, wherein the HGF receptor or protein functionally equivalent to an HGF receptor is selected from the following (i) to (iv):
(i) a protein comprising the amino acid sequence of SEQ ID NO: 4;
(ii) a protein encoded by a nucleic acid comprising the coding region of the nucleotide sequence of SEQ ID NO: 3;
(iii) a protein comprising an amino acid sequence with a substitution, deletion, insertion, and/or addition of one or more amino acids in the amino acid sequence of SEQ ID NO: 4, which is functionally equivalent to a protein comprising the amino acid sequence of SEQ ID NO: 4; and
(iv) a protein encoded by a nucleic acid that hybridizes under stringent conditions to a nucleic acid comprising the nucleotide sequence of SEQ ID NO: 3, which is functionally equivalent to a protein comprising the amino acid sequence of SEQ ID NO: 4.

29. A method of screening for a compound having lymphangiogenesis-promoting activity or a compound having an effect of preventing or treating lymphedema, wherein the method comprises the following steps:
(a) contacting a test compound with a cell expressing an HGF receptor or a protein functionally equivalent to an HGF receptor;
(b) measuring the growth capacity or migratory activity of the cell, or phosphorylation of a signaling molecule; and
(c) selecting a test compound that increases the growth capacity or migratory activity of the cell, or causes phosphorylation of the signaling molecule, as compared to when the test compound is not contacted.

30. The method of claim 29, wherein the HGF receptor or protein functionally equivalent to an HGF receptor is selected from the following (i) to (iv):
(i) a protein comprising the amino acid sequence of SEQ ID NO: 4;
(ii) a protein encoded by a nucleic acid comprising the coding region of the nucleotide of SEQ ID NO: 3;
(iii) a protein comprising an amino acid sequence with a substitution, deletion, insertion, and/or addition of one or more amino acids in the amino acid sequence of SEQ ID NO: 4, which is functionally equivalent to a protein comprising the amino acid sequence of SEQ ID NO: 4; and
(iv) a protein encoded by a nucleic acid that hybridizes under stringent conditions to a nucleic acid comprising the nucleotide sequence of SEQ ID NO: 3, which is functionally equivalent to a protein comprising the amino acid sequence of SEQ ID NO: 4.

31.-38. (canceled)

39. A method for preventing or treating lymphedema, which comprises the step of administering, in a naked state, a mammalian expression vector into which a nucleic acid encoding an HGF or a protein functionally equivalent to an HGF has been inserted, to or around an affected area in a subject by intramuscular injection.

40. The method of claim 39, wherein the nucleic acid encoding an HGF or a protein functionally equivalent to an HGF is a nucleic acid encoding any one of the following (a) to (d):
(a) a protein comprising the amino acid sequence of SEQ ID NO: 2;
(b) a protein encoded by a nucleic acid comprising the coding region of the nucleotide sequence of SEQ ID NO: 1;
(c) a protein comprising an amino acid sequence with a substitution, deletion, insertion, and/or addition of one or more amino acids in the amino acid sequence of SEQ ID NO: 2, which is functionally equivalent to a protein comprising the amino acid sequence of SEQ ID NO: 2; and
(d) a protein encoded by a nucleic acid that hybridizes under stringent conditions to a nucleic acid comprising the nucleotide sequence of SEQ ID NO: 1, which is functionally equivalent to a protein comprising the amino acid sequence of SEQ ID NO: 2.

41. The method of claim 40, wherein the vector comprises the nucleotide sequence of SEQ ID NO: 5 or 6.

42.-44. (canceled)

45. A method for treating lymphedema in a human subject, wherein the method comprises:
preparing a naked plasmid vector comprising the nucleotide sequence of SEQ ID NO: 5;
determining that a human subject suffers from lymphedema, and is in need of treating said lymphedema;
identifying a site of lymphedema characterized by the occlusion of lymphatic vessels in the subject;
injecting the naked plasmid vector into a muscle at or around the site of lymphedema such that growth and migration of lymphatic endothelial cells is promoted at or around the site of lymphedema;
thereby increasing lymphangiogenesis and the number of lymphatic vessels, and decreasing the fluid associated with lymphedema resulting from occlusion of lymphatic vessels in the subject.

46. The method of claim 45, wherein the naked nucleic acid consists of the nucleotide sequence of SEQ ID NO: 5.
